(19) United States
(12) Patent Application Publication
(10) Pub. No.: US 2025/0186556 A1
(43) Pub. Date: Jun. 12, 2025

(54) COMBINATION THERAPY WITH IMMUNOMODULATORS, DYRK1A INHIBITORS, AND GLP1R AGONISTS FOR TYPE 1 DIABETES TREATMENT

(71) Applicant: ICAHN SCHOOL OF MEDICINE AT MOUNT SINAI, New York, NY (US)

(72) Inventors: Adolfo GARCIA-OCANA, New York, NY (US); Andrew F. STEWART, New York, NY (US); Robert J. DEVITA, New York, NY (US); Geming LU, New York, NY (US); Peng WANG, New York, NY (US)

(21) Appl. No.: 18/867,399

(22) PCT Filed: Jun. 1, 2023
(86) PCT No.: PCT/US2023/024153

§ 371 Date: Nov. 19, 2024

Related U.S. Application Data
(60) Provisional application No. 63/347,977, filed on Jun. 1, 2022.

(51) Int. Cl. A61K 38/26; A61K 31/437; A61K 39/395; A61P 3/10

(52) U.S. Cl. CPC A61K 38/26; A61K 31/437; A61K 39/3955; A61P 3/10

(57) ABSTRACT

Disclosed herein are methods of treating a subject for a condition associated with insufficient insulin secretion by administering to a subject in need of treatment for a condition associated with an insufficient level of insulin secretion a dual-specificity tyrosine phosphorylation-regulated kinase 1A (DYRK1A) inhibitor, a glucagon-like peptide-1 receptor (GLP1R) agonist, and an immunomodulatory monoclonal antibody and/or an immunosuppressive agent (e.g., anti-CD3 antibody), where said administering is carried out under conditions effective to reverse loss of β-cell mass and function in the subject to treat the subject for the condition associate with insufficient insulin secretion. Also disclosed is a composition and a method of increasing β-cell mass and function in a population of pancreatic beta cells.

This application claims the priority benefit of U.S. Provisional Application Ser. No. 63/347,977, filed Jun. 1, 2022, which is hereby incorporated by reference in its entirety.

This invention was made with government support under DK105015 awarded by the National Institute of Health. The government has certain rights in the invention.

### FIELD

Described are methods and compositions for treating a subject for a condition associated with insufficient insulin secretion.

### BACKGROUND

1.6 million people in the U.S. and 20 million in the world have Type 1 diabetes (“T1D”). Approximately twice as many are family members at high risk of developing T1D. T1D results from autoreactive host immune cells (T-cell and other immune system cells) damaging and killing insulin-producing β-cells in the pancreas. The etiology of this is incompletely understood, but involves combinations of genetic, environmental, and infectious agent factors. There is no known effective therapy that can delay, prevent, or reverse β-cell loss and progression to diabetes in people with T1D. Progress is being made in immunomodulatory therapies that can block or delay immune system dysfunction in people in the early stages of T1D who still have an ample supply of β-cells, but these therapies do not restore lost β-cells in people with established T1D. This β-cell loss in T1D inspires attempts to replace lost β-cells through pancreas transplant, islet transplant, and transplant of stem cell-derived human β-cells, but these are not scalable or cost-effective approaches for millions of people with T1D.

A failure in self-tolerance leads to autoimmune destruction of pancreatic β-cells and T1D. Therapies able to simultaneously 1) modulate T-cell activation; 2) protect β-cells against known drivers of T1D-associated β-cell dysfunction and destruction, such as proinflammatory cytokines and ER stress; and, 3) induce β-cell regeneration, could provide a therapeutic approach for curing T1D.

It has recently been shown that combined treatment for three months with the DYRK1A inhibitor harmine, and the GLP1R agonist exendin-4, increases human β-cell mass by 6-7-fold in streptozotocin-induced diabetic mice transplanted with human islets (Rosselot et al., “Human Beta Cell Mass Expansion In Vivo with a Harmine and Exendin-4 Combination: Quantification and Visualization by iDISCO+ 3D Imaging,” biorxiv (2021)). If translated to T1D patients, this remarkable increase in human β-cell mass could be enough to normalize blood glucose levels. However, T1D associated autoimmunity will likely continue to destroy newly regenerated β-cells. Interestingly, several immune modulation interventions have been shown to delay the decline in β-cell function in patients with recent-onset T1D (Atkinson et al., “The Challenge of Modulating j-Cell Autoimmunity in Type 1 Diabetes,” Lancet Diabetes Endocrinol. 7:52-64 (2019)). One such promising therapy is a humanized, non-mitogenic aglycosylated anti-CD3 monoclonal antibody, teplizumab. This anti-CD3 antibody reduces the loss of β-cell function in patients with new-onset type 1 diabetes for up to seven years following initial diagnosis (Herold et al., “Teplizumab (anti-CD3 mAb) Treatment Preserves C-Peptide Responses in Patients with New-Onset Type 1 Diabetes In a Randomized Controlled Trial: Metabolic and Immunologic Features at Baseline Identify a Subgroup of Responders,” Diabetes 62:3766-74 (2013); Herold et al., “Anti-CD3 Monoclonal Antibody in New-Onset Type 1 Diabetes Mellitus,” N. Engl. J. Med. 346:1692-8 (2002); Keymeulen et al., “Insulin Needs After CD3-Antibody Therapy in New-Onset Type 1 Diabetes,” N. Engl. J. Med. 352:2598-608 (2005); Sherry et al., “Teplizumab for Treatment of Type 1 Diabetes (Protégé Study): 1-Year Results from a Randomised, Placebo-Controlled Trial,” Lancet 378:487-97 (2011); Hagopian et al., “Teplizumab Preserves C-Peptide in Recent-Onset Type 1 Diabetes: Two-Year Results from the Randomized, Placebo-Controlled Protégé Trial,” Diabetes 62:3901-8 (2013); Herold et al., “An Anti-CD3 Antibody, Teplizumab, In Relatives at Risk for Type 1 Diabetes,” N. Engl. J. Med. 381:603-613 (2019)). The antibody modifies autoreactive CD8+T-lymphocytes, important effector cells that kill β-cells in T1D. Subsequent reports suggest that the CD3-specific monoclonal antibodies enhance the function and/or proliferation of regulatory T-cells, which then exert a dominant suppression on autoreactive T-cells. While this is an exciting advance in the treatment of T1D, further improvement is required in several areas. First, this approach only delays T1D onset but does not cure T1D; second, the effect on established T1D subjects is limited probably reflecting the facts that β-cell mass is reduced in these patients, and that β-cells are unable to replicate or regenerate; and, third, there are potential associated risks with the use of high doses of this antibody that relate to lymphoproliferative disorders in younger patients.

The present disclosure is directed to overcoming deficiencies in the art.

### SUMMARY

One aspect of the present disclosure is directed to a method of treating a subject for a condition associated with insufficient insulin secretion. This method involves administering to a subject in need of treatment for a condition associated with an insufficient level of insulin secretion a dual-specificity tyrosine phosphorylation-regulated kinase 1A (DYRK1A) inhibitor, a glucagon-like peptide-1 receptor (GLP1R) agonist, and an immunomodulatory monoclonal antibody, optionally wherein the immunomodulatory monoclonal antibody is an anti-CD3 antibody, where said administering is carried out under conditions effective to reverse loss of β-cell mass and function in the subject to treat the subject for the condition associate with insufficient insulin secretion.

Another aspect of the present disclosure relates to a composition comprising a dual-specificity tyrosine phosphorylation-regulated kinase 1A (DYRK1A) inhibitor, a glucagon-like peptide-1 receptor (GLP1R) agonist, and an immunomodulatory monoclonal antibody and/or an immunosuppressive agent (e.g., anti-CD3 antibody).

A further aspect of the present disclosure relates to a method of increasing β-cell mass and function in a population of pancreatic beta cells. This method involves contacting a population of pancreatic beta cells with a dual-specificity tyrosine phosphorylation-regulated kinase 1A (DYRK1A) inhibitor, a glucagon-like peptide-1 receptor (GLP1R) agonist, and a low dose of an immunomodulatory monoclonal antibody and/or an immunosuppressive agent (e.g., an anti-CD3 antibody), where said contacting is carried out under conditions effective to increase β-cell mass and function in the population of pancreatic beta cells.

In the examples of the present disclosure it was hypothesized that combination treatment employing an anti-CD3 antibody with the β-cell regenerative drugs harmine and exendin-4 would reverse T1D. To test this hypothesis, a mouse model that spontaneously develops T1D, the NOD female mouse, was treated with anti-CD3 monoclonal antibody for three consecutive days followed by continuous delivery of harmine plus exendin-4 for eight weeks. It was surprisingly found that this treatment combination completely reversed T1D in 100% of mice. These mice show reduced insulitis in the pancreas, enhanced β-cell proliferation and mass, reduced activated T-cells (Th1+ cells), and increased regulatory T-cells.

The examples described herein demonstrate for the first time that an anti-CD3 immunomodulatory treatment (e.g., at a low dose), together with harmine+exendin-4 β-cell regenerative and anti-apoptotic combination treatment increases immune tolerance, enhances (3-cell proliferation and mass, and protects β-cells from cytokines and ER stress, effects collectively leading to reversal of early onset T1D in a mouse model of T1D.

The present disclosure relates to a therapeutically and financially effective combination of immunomodulatory and β-cell regenerative therapy that can be applied to millions of people with established, long term T1D, and with recent onset T1D.

### BRIEF DESCRIPTION OF THE DRAWINGS

FIGS. 1A-1H show the protective effect of harmine plus exendin-4 on human 0-cells in vitro after treatment with two canonical inducers of β-cell death associated with T1D, proinflammatory cytokines and ER stress. FIG. 1A is a graph showing results of human islet cells in culture treated with cytokines (CKS) and with or without 10 μM harmine (H), 10 nM exendin-4 (E), or both (H+E) for 24 hours. N=7 different human islet preparations were analyzed. FIG. 1B shows representative images of those experiments. FIG. 1C is a graph showing the results of human islet cells treated with 500 nM thapsigargin (TH) and with or without E, H, or E+H for 24 hours. N=5 different human islet preparations analyzed. P values are indicated in the figure; each dot represents a different human islet preparation. FIGS. 1D-1H are graphs showing single cell RNA sequencing (scRNA-seq) analysis of human islets treated with cytokines and H, E, and H+E as in FIG. 1A. Gene set enrichment analysis (GSEA) of human β-cells in the scRNA-seq studies reveals an increase in pro-inflammatory (FIG. 1D), intrinsic and extrinsic apoptosis (FIG. 1E), human leukocyte antigen (HLA) class 1 molecules (FIG. 1F), chemokines CXCL9-11 (FIG. 1G), and interferon regulator factor 1-9 (FIG. 1H) signaling pathways with cytokine treatment that are highly reduced towards control expression after treatment with H+E but not with the drugs alone (except the inhibitory HLA-E that is increased by H and H+E). These are the results of N=3 different human islet preparations. *P<0.05, **P<0.01, ***P<0.001.

FIGS. 2A-2F show that treatment with harmine and exendin-4 after administration of anti-CD3 reverses diabetes in NOD diabetic mice. FIGS. 2A-2F are graphs showing blood glucose levels in mice before and after becoming spontaneously diabetic and then treated with 5 μg of. IgG per mouse per day for three days and 3 mg/kg/day harmine (H) and 0.1 mg/kg/day exendin-4 (E), or vehicle (H₂O) for eight weeks (N=6-8 mice) (FIG. 2A); anti-CD3 per mouse per day for three days and 3 mg/kg/day harmine (H) or 0.1 mg/kg/day exendin-4 (E) for eight weeks (N=5-6 (FIG. 2B); anti-CD3 per day for three days and 3 mg/kg/day harmine (H) and 0.1 mg/kg/day exendin-4 (E), or vehicle (H₂O) for eight weeks (N=19 per group) (FIG. 2C). One alzet minipump (pump) was implanted every four weeks. FIGS. 2D-2F are graphs showing percentage of diabetic mice after treatments as shown in FIG. 2A (FIG. 2D), FIG. 2B (FIG. 2E), and FIG. 2C (FIG. 2F). Diabetes is defined as blood glucose above 250 mg/dl.

FIGS. 3A-3J show the results of immune profiling in splenocytes from NOD mice treated with anti-CD3 and harmine plus exendin-4. FIG. 3A is a graph showing the total number of CD45⁺ cells. FIG. 3B is a graph showing the ratio of CD4/CD8 T cells. FIG. 3C is a graph showing CD44/CD62L (naïve, memory, and effector) CD8⁺ cells. FIGS. 3D-3E are graphs showing that activated (Interferon-gamma, IFNg⁺ cells, Th1) CD4⁺ and CD8⁺ lymphocytes are decreased with anti-CD3 and H+E treatment. FIGS. 3F-3G are graphs showing FoxP3⁺CD25⁺ (Tregs) cells are increased in the spleen of mice treated with anti-CD3 and H+E. FIG. 3H is a graph representing circulating TNF alpha levels in representative mice (N=5) from FIG. 2C. FIG. 3I are graphs showing the levels of CXCR3 expression in CD4⁺ and CD8⁺ cells in representative mice (N=3) from FIG. 2C. FIG. 3J are graphs showing the levels of T cell exhaustion markers PD1, TIGIT, TOX, and EOMES in CD4⁺ and CD8⁺ cells in NOD mice treated for three days with anti-CD3 and then for 2-weeks with vehicle (water) or H+E as in FIG. 2C (N=3 to 7 mice). *P<0.05; **P<0.01.

FIGS. 4A-4E show the results of pancreas analysis in NOD mice treated with anti-CD3 and harmine (H) plus exendin-4 (E). FIG. 4A shows representative images of hematoxylin & eosin staining of pancreases from mice treated in FIG. 2C. Top image is from a mouse treated with vehicle (water) and bottom image is from a mouse treated with H+E. FIG. 4B is a graph showing quantitation of insulitis scores in islets of these mice (N=19 mice per group). Score 0, no insulitis; score 1, peri-insulitis; score 2, mild insulitis; score 3, strong insulitis; score 4, close to 100% insulitis. FIG. 4C shows flow cytometry analysis of islets from 3 mice of these groups of mice to detect CD45⁺ cells (immune cells). FIG. 4D is a graph showing quantitation of Ki67⁺ (red)/insulin⁺ (green) cells/DAPI (blue, Nuclei) (β-cell proliferation), and TUNEL (green), insulin (red), and DAPI (blue, nuclei) (β-cell death) in pancreatic sections from the mice treated in FIG. 2C. Arrows indicate Ki67⁺/insulin⁺ cells (upper images) or TUNEL⁺insulin⁺ cells (lower images). Graphs with quantitation of these mice appear on the right. FIG. 4E is a graph showing quantitation of β-cell mass (mg). N=6-19 mice per group. *P<0.05; **P<0.01: ***P<0.001; ****P<0.00001.

### DETAILED DESCRIPTION

Disclosed are methods and compositions for treating a subject for a condition associated with insufficient insulin secretion.

One aspect of the present disclosure is directed to a method of treating a subject for a condition associated with insufficient insulin secretion. This method involves administering to a subject in need of treatment for a condition associated with an insufficient level of insulin secretion a dual-specificity tyrosine phosphorylation-regulated kinase 1A (DYRK1A) inhibitor, a glucagon-like peptide-1 receptor (GLP1R) agonist, and an immunomodulatory monoclonal antibody (e.g., an anti-CD3 antibody), where said administering is carried out under conditions effective to reverse loss of β-cell mass and function in the subject to treat the subject for the condition associate with insufficient insulin secretion.

Suitable DYRK1A inhibitors and GLP1R agonists for carrying out methods of the present disclosure are described in International Publication No. WO 2018/081401 to Stewart et al., International Publication No. 2019/100062 to DeVita et al., International Publication No. WO 2019/183245 to Kumar et al., International Publication No. WO 2019/100062 to DeVita et al., International Publication No. WO 2019/136320 to Stewart et al., International Publication No. WO 2020/142485 to DeVita et al., International Publication No. WO 2020/142486 to DeVita et al., and International Publication No. WO 2021/263129 to DeVita et al., which are hereby incorporated by reference in their entirety.

Several DYRK1A inhibitors from natural sources as well as small molecule drug discovery programs have been identified and characterized, and may be used in carrying methods disclosed herein. For example, suitable DYRK1A inhibitors include, without limitation, harmine; INDY (having the chemical structure

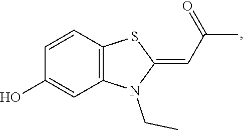 [SMILES: CCN1/C(=C/C(C)=O)Sc2ccc(O)cc21]

as described in Wang et al., “A High-Throughput Chemical Screen Reveals That Harmine-Mediated Inhibition of DYRK1A Increases Human Pancreatic Beta Cell Replication,” Nature Medicine 21:383-388 (2015), which is hereby incorporated by reference in its entirety); leucettine, having the chemical structure

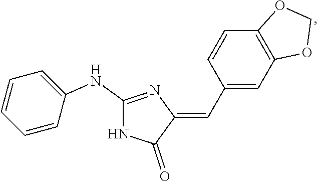 [SMILES: O=C1NC(Nc2ccccc2)=N/C1=C\c1ccc2c(c1)OCO2]

as described in Tahtouh et al., “Selectivity, Cocrystal Structures, and Neuroprotective Properties of Leucettines, a Family of Protein Kinase Inhibitors Derived from the Marine Sponge Alkaloid Leucettamine B,” J. Med. Chem. 55:9312-30 (2012), which is hereby incorporated by reference in its entirety); 5-iodotubercidin (5-IT); GNF4877; harmine analogs; CC-401; thiadiazine kinase inhibitors; and others. Additional suitable DYRK1A inhibitors include, but are not limited to, GNF7156 and GNF6324 (see Shen et al., “Inhibition of DYRK1A and GSK3B Induces Human Beta Cell Proliferation,” Nat. Commun. 6:8372 (2015), which is hereby incorporated by reference in its entirety). In carrying out the methods of the present disclosure or forming the compositions of the present disclosure, combinations of DYRK1A inhibitors may be used. Among all the DYRK1A inhibitors, harmine and its analogues (β-carbolines) are the most commonly studied and remain the most potent and orally bioavailable class of inhibitors covered to date (Becker et al., “Activation, Regulation, and Inhibition of DYRK1A,” FEBS J. 278(2):246-256 (2011) and Smith et al., “Recent Advances in the Design, Synthesis, and Biological Evaluation of Selective DYRK1A Inhibitors: A New Avenue for a Disease Modifying Treatment of Alzheimer's?,” ACS Chem. Neurosci. 3(11):857-872 (2012), which are hereby incorporated by reference in their entirety).

Apart from harmine, EGCg and other flavan-3-ols (Guedj et al., “Green Tea Polyphenols Rescue of Brain Defects Induced by Overexpression of DYRK1A,” PLoS One 4(2):e4606 (2009) and Bain et al., “The Specificities of Protein Kinase Inhibitors: An Update,” Biochem. J. 371(1):199-204 (2003), which are hereby incorporated by reference in their entirety), leucettines (Tahtouh et al., “Selectivity, Cocrystal Structures, and Neuroprotective Properties of Leucettines, a Family of Protein Kinase Inhibitors Derived from the Marine Sponge Alkaloid Leucettamine B,” J. Med. Chem. 55(21):9312-9330 (2012) and Naert et al., “Leucettine L41, a DYRK1A-preferential DYRKs/CLKs Inhibitor, Prevents Memory Impairments and Neurotoxicity Induced by Oligomeric Aβ25-35 Peptide Administration in Mice,” Eur. Neuropsychopharmacol. 25(11):2170-2182 (2015), which are hereby incorporated by reference in their entirety), quinalizarine (Cozza et al., “Quinalizarin as a Potent, Selective and Cell-permeable Inhibitor of Protein Kinase CK2,” Biochem. J. 421(3):387-395 (2009), which is hereby incorporated by reference in its entirety), peltogynoids Acanilol A and B (Ahmadu et al, “Two New Peltogynoids from Acacia nilotica Delile with Kinase Inhibitory Activity,” Planta Med. 76(5):458-460 (2010), which is hereby incorporated by reference in its entirety), benzocoumarins (dNBC) (Sarno et al., “Structural Features Underlying the Selectivity of the Kinase Inhibitors NBC and dNBC: Role of a Nitro Group that Discriminates Between CK2 and DYRK1A,” Cell. Mol. Life Sci. 69(3):449-460 (2012), which is hereby incorporated by reference in its entirety), and indolocarbazoles (Starosporine, rebeccamycin and their analogues) (Sanchez et al., “Generation of Potent and Selective Kinase Inhibitors by Combinatorial Biosynthesis of Glycosylated Indolocarbazoles,” Chem. Commun. 27:4118-4120 (2009), which is hereby incorporated by reference in its entirety), are other natural products that have been shown to inhibit DYRK1A and other kinases.

Among the other scaffolds identified from small molecule drug discovery attempts, INDY (Ogawa et al., “Development of a Novel Selective Inhibitor of the Down Syndrome-Related Kinase DyrklA,” Nat. Commun. 1: Article Number 86 (2010), which is hereby incorporated by reference in its entirety), DANDY (Gourdain et al., “Development of DANDYs, New 3,5-Diaryl-7-Azaindoles Demonstrating Potent DYRK1A Kinase Inhibitory Activity,” J. Med Chem. 56(23):9569-9585 (2013), which is hereby incorporated by reference in its entirety), and FINDY (Kii et al., “Selective Inhibition of the Kinase DYRK1A by Targeting its Folding Process,” Nat. Commun. 7:11391 (2016), which is hereby incorporated by reference in its entirety), pyrazolidine-diones (Koo et al., “QSAR Analysis of Pyrazolidine-3,5-Diones Derivatives as DyrklA Inhibitors,” Bioorg. Med Chem. Lett. 19(8):2324-2328 (2009); Kim et al., “Putative Therapeutic Agents for the Learning and Memory Deficits of People with Down Syndrome,” Bioorg. Med Chem. Lett. 16(14):3772-3776 (2006), which are hereby incorporated by reference in their entirety), amino-quinazolines (Rosenthal et al., “Potent and Selective Small Molecule Inhibitors of Specific Isoforms of Cdc2-Like Kinases (Clk) and Dual Specificity Tyrosine-Phosphorylation-Regulated Kinases (Dyrk),” Bioorg. Med Chem. Lett. 21(10):3152-3158 (2011), which is hereby incorporated by reference in its entirety), meriolins (Giraud et al., “Synthesis, Protein Kinase Inhibitory Potencies, and In Vitro Antiproliferative Activities of Meridianin Derivatives,” J. Med Chem. 54(13):4474-4489 (2011); Echalier et al., “Meriolins (3-(Pyrimidin-4-yl)-7-Azaindoles): Synthesis, Kinase Inhibitory Activity, Cellular Effects, and Structure of a CDK2/Cyclin A/Meriolin Complex,” J. Med Chem. 51(4):737-751 (2008); and Akue-Gedu et al., “Synthesis and Biological Activities of Aminopyrimidyl-Indoles Structurally Related to Meridianins,” Bioorg. Med Chem. 17(13):4420-4424 (2009), which are hereby incorporated by reference in their entirety), pyridine and pyrazines (Kassis et al., “Synthesis and Biological Evaluation of New 3-(6-hydroxyindol-2-yl)-5-(Phenyl) Pyridine or Pyrazine V-Shaped Molecules as Kinase Inhibitors and Cytotoxic Agents,” Eur. J. Med Chem. 46(11):5416-5434 (2011), which is hereby incorporated by reference in its entirety), chromenoidoles (Neagoie et al., “Synthesis of Chromeno[3,4-b]indoles as Lamellarin D Analogues: A Novel DYRK1A Inhibitor Class,” Eur. J. Med Chem. 49:379-396 (2012), which is hereby incorporated by reference in its entirety), 11H-indolo[3,2-c]quinoline-6-carboxylic acids, thiazolo[5,4-f]quinazolines (EHT 5372) (Foucourt et al., “Design and Synthesis of Thiazolo[5,4-f]quinazolines as DYRK1A Inhibitors, Part I.,” Molecules 19(10):15546-15571 (2014) and Coutadeur et al., “A Novel DYRK1A (Dual Specificity Tyrosine Phosphorylation-Regulated Kinase 1A) Inhibitor for the Treatment of Alzheimer's Disease: Effect on Tau and Amyloid Pathologies In Vitro,” J. Neurochem. 133(3):440-451 (2015), which are hereby incorporated by reference in their entirety), and 5-iodotubercidin (Dirice et al., “Inhibition of DYRK1A Stimulates Human Beta Cell Proliferation,” Diabetes 65(6):1660-1671 (2016) and Annes et al., “Adenosine Kinase Inhibition Selectively Promotes Rodent and Porcine Islet β-cell Replication,” Proc. Natl. Acad. Sci. 109(10):3915-3920 (2012), which are hereby incorporated by reference in their entirety) show potent DYRK1A activity with varying degrees of kinase selectivity.

Additional suitable DYRK1A inhibitors include, without limitation, GNF2133 (Liu et al., “Selective DYRK1A Inhibitor for the Treatment of Type 1 Diabetes: Discovery of 6-Azaindole Derivative GNF2133,” J. Med. Chem. 63:2958-2973 (2020), which is hereby incorporated by reference in its entirety) as well as those described in Liu et al., “DYRK1A Inhibitors for Disease Therapy: Current Status and Perspectives,” Eur. J. Med. Chem. 229:114062 (2022), which is hereby incorporated by reference in its entirety).

Suitable thiadiazine kinase inhibitors include, for example and without limitation, those described in International Publication No. WO 2019/100062 to DeVita et al. and International Publication No. WO 2019/136320 to Stewart et al. (see Tables 1 and 2), which are hereby incorporated by reference in their entirety.

As described supra, glucagon-like peptide-1 receptor agonists mimic the effects of the incretin hormone GLP-1, which is released from the intestine in response to food intake. Their effects include increasing insulin secretion, decreasing glucagon release, increasing satiety, and slowing gastric emptying.

Suitable GLP1R agonists for carrying out the methods disclosed herein are described in PCT Publication No. WO 2019/136320 to Stewart et al., which is hereby incorporated by reference in its entirety, and include, without limitation, exenatide, liraglutide, exenatide LAR, taspoglutide, lixisenatide, albiglutide, dulaglutide, and semaglutide. Exenatide and Exenatide LAR are synthetic exendin-4 analogues obtained from the saliva of the Heloderma suspectum (lizard). Liraglutide is an acylated analogue of GLP-1 that self-associates into a heptameric structure that delays absorption from the subcutaneous injection site. Taspoglutide shares 3% homology with the native GLP-1 and is fully resistant to DPP-4 degradation. Lixisenatide is a human GLP1R agonist. Albiglutide is a long-acting GLP-1 mimetic, resistant to DPP-4 degradation. Dulaglutide is a long-acting GLP1 analogue. Semaglutide is a GLP1R agonist approved for the use of T2D. Clinically available GLP1R agonists include, e.g., exenatide, liraglutide, albiglutide, dulaglutide, lixisenatide, semaglutide.

In some embodiments, the GLP1R agonist is selected from the group consisting of exendin-4, GLP1(7-36), liraglutide, lixisenatide, semaglutide, tirzepatide (also known as Mounjaro) and combinations thereof.

Additional suitable GLP1 agonists include, without limitation, disubstituted-7-aryl-5,5-bis(trifluoromethyl)-5,8-dihydropyrimido[4,5-d]pyrimidine-2,4(1H,3H)-dione compounds and derivatives thereof, e.g., 7-(4-Chlorophenyl)-1,3-dimethyl-5,5-bis(trifluoromethyl)-5,8-dihydropyrimido[4,5-d]pyrimidine-2,4(1H,3H)-dione (see, e.g., Nance et al., “Discovery of a Novel Series of Orally Bioavailable and CNS Penetrant Glucagon-like Peptide-1 Receptor (GLP-1R) Noncompetitive Antagonists Based on a 1,3-Disubstituted-7-aryl 5,5-bis(trifluoromethyl)-5,8-dihydropyrimido[4,5-d]pyrimidine-2,4(1H,3H)-dione Core,” J. Med. Chem. 60:1611-1616 (2017), which is hereby incorporated by reference in its entirety).

Further suitable GLP1 agonists include positive allosteric modulators (“PAMS”) of GLP1R, e.g., (S)-2-cyclopentyl-N-((1-isopropylpyrrolidin-2-yl)methyl)-10-methyl-1-oxo-1,2-dihydropyrazino[1,2-a]indole-4-carboxamide; (R)-2-cyclopentyl-N-((1-isopropylpyrrolidin-2-yl)methyl)-10-methyl-1-oxo-1,2-dihydropyrazino[1,2-a]indole-4-carboxamide; 2-cyclopentyl-N-(((S)-1-isopropylpyrrolidin-2-yl)methyl)-10-methyl-1-oxo-1,2,3,4-tetrahydropyrazino[1,2-a]indole-4-carboxamide; N-(((S)-1-isopropylpyrrolidin-2-yl)methyl)-10-methyl-1-oxo-2-((S)-tetrahydrofuran-3-yl)-1,2-dihydropyrazino[1,2-a]indole-4-carboxamide; N-(((R)-1-isopropylpyrrolidin-2-yl)methyl)-10-methyl-1-oxo-2-((S)-tetrahydrofuran-3-yl)-1,2-dihydropyrazino[1,2-a]indole-4-carboxamide; (S)-2-cyclopentyl-8-fluoro-N-((1-isopropylpyrrolidin-2-yl)methyl)-10-methyl-1-oxo-1,2-dihydropyrazino[1,2-a]indole-4-carboxamide; (R)-2-cyclopentyl-8-fluoro-N-((1-isopropylpyrrolidin-2-yl)methyl)-10-methyl-1-oxo-1,2-dihydropyrazino[1,2-a]indole-4-carboxamide; (R)-2-cyclopentyl-N-(((S)-1-isopropylpyrrolidin-2-yl)methyl)-10-methyl-1-oxo-1,2,3,4-tetrahydropyrazino[1,2-a]indole-4-carboxamide; (S)-2-cyclopentyl-N-(((S)-1-isopropylpyrrolidin-2-yl)methyl)-10-methyl-1-oxo-1,2,3,4-tetrahydropyrazino[1,2-a]indole-4-carboxamide; (S)-10-chloro-2-cyclopentyl-N-((1-isopropylpyrrolidin-2-yl)methyl)-1-oxo-1,2-dihydropyrazino[1,2-a]indole-4-carboxamide; (R)-10-chloro-2-cyclopentyl-N-((1-isopropylpyrrolidin-2-yl)methyl)-1-oxo-1,2-dihydropyrazino[1,2-a]indole-4-carboxamide; (S)-10-bromo-2-cyclopentyl-N-((1-isopropylpyrrolidin-2-yl)methyl)-1-oxo-1,2-dihydropyrazino[1,2-a]indole-4-carboxamide; (R)-10-bromo-2-cyclopentyl-N-((1-isopropylpyrrolidin-2-yl)methyl)-1-oxo-1,2-dihydropyrazino[1,2-a]indole-4-carboxamide; (R)-N-((1-isopropylpyrrolidin-2-yl)methyl)-10-methyl-1-oxo-2-phenyl-1,2-dihydropyrazino[1,2-a]indole-4-carboxamide; (S)-10-cyano-2-cyclopentyl-N-((1-isopropylpyrrolidin-2-yl)methyl)-1-oxo-1,2-dihydropyrazino[1,2-a]indole-4-carboxamide; (S)-2-cyclopentyl-N-((1-isopropylpyrrolidin-2-yl)methyl)-1-oxo-10-vinyl-1,2-dihydropyrazino[1,2-a]indole-4-carboxamide; (S)-N-((1-isopropylpyrrolidin-2-yl)methyl)-10-methyl-2-(1-methyl-1H-pyrazol-4-yl)-1-oxo-1,2-dihydropyrazino[1,2-a]indole-4-carboxamide; (R)-N-((1-isopropylpyrrolidin-2-yl)methyl)-10-methyl-2-(1-methyl-1H-pyrazol-4-yl)-1-oxo-1,2-dihydropyrazino[1,2-a]indole-4-carboxamide; (S)-N-((1-isopropylpyrrolidin-2-yl)methyl)-10-methyl-1-oxo-2-(pyridin-3-yl)-1,2-dihydropyrazino[1,2-a]indole-4-carboxamide; (R)-N-((1-isopropylpyrrolidin-2-yl)methyl)-10-methyl-1-oxo-2-(pyridin-3-yl)-1,2-dihydropyrazino[1,2-a]indole-4-carboxamide; N-(azetidin-2-ylmethyl)-2-cyclopentyl-10-methyl-1-oxo-1,2-dihydropyrazino[1,2-a]indole-4-carboxamide; and 2-cyclopentyl-N-((1-isopropylazetidin-2-yl)methyl)-10-methyl-1-oxo-1,2-dihydropyrazino[1,2-a]indole-4-carboxamide; or pharmaceutically acceptable salts thereof (see PCT Publication No. WO 2017/117556, which is hereby incorporated by reference in its entirety).

Additional suitable GLP1 agonists include, without limitation, chimeric peptides such as a dual glucose-dependent insulinotropic polypeptide (GIP) and glucagon-like peptide-1 (GLP-1) receptor agonist (i.e., tirzepatide). See, e.g., Frias et al., “Tirzepatide Versus Semaglutide Once Weekly in Patients with Type 2 Diabetes,” N. Engl. J. Med. 385:503-515 (2021), which is hereby incorporated by reference in its entirety. Both GIP and GLP1 are GPCRs that act via cAMP. Thus, in some embodiments, the GLP1 agonists increase cAMP in a population of beta cells (e.g., human beta cells).

Glucagon-like peptide-1 receptor agonists mimic the effects of the incretin hormone GLP-1, which is released from the intestine in response to food intake. Their effects include increasing insulin secretion, decreasing glucagon release, increasing satiety, and slowing gastric emptying. An alternate approach to enhancing GLP1 concentrations in blood is prevention of its degradation by the enzyme DPP4. The GLP1 receptor agonists and the DDP4 inhibitors are among the most widely used drugs for the treatment of Type 2 diabetes (Campbell et al., “Pharmacology, Physiology and Mechanisms of Incretin Hormone Action,” Cell Metab. 17:819-37 (2013); Guo X-H., “The Value of Short- and Long-Acting Glucagon-Like Peptide Agonists in the Management of Type 2 Diabetes Mellitus: Experience with Exenatide,” Curr. Med. Res. Opinion 32(1):61-76 (2016); Deacon et al., “Dipeptidyl Peptidase-4 Inhibitors for the Treatment of Type 2 Diabetes: Comparison, Efficacy and Safety,” Expert Opinion on Pharmacotherapy 14:2047-58 (2013); Lovshin, “Glucagon-Like Peptide-1 Receptor Agonists: A Class Update for Treating Type 2 Diabetes,” Can. J. Diabetes 41:524-35 (2017); and Yang et al., “Lixisenatide Accelerates Restoration of Normoglycemia and Improves Human Beta Cell Function and Survival in Diabetic Immunodeficient NOD-scid IL2rg(null) RIP-DTR Mice Engrafted With Human Islets,” Diabetes Metab. Syndr. Obes. 8:387-98 (2015), which are hereby incorporated by reference in their entirety).

Thus, in addition to or in place of a GLP1 agonist, the methods and compositions according to the present disclosure, may include a Dipeptidyl Peptidase IV (DDP4) inhibitor. Exemplary suitable DDP4 inhibitors include, without limitation, sitagliptin, vildagliptin, saxagliptin, alogliptin, teneligliptin, and anagliptin.

In carrying out the methods disclosed herein, the immunomodulatory monoclonal antibody may be an anti-CD3 antibody. A suitable anti-CD3 antibody can include any antibody directed against or that can specifically bind the CD3 receptor on the surface of T cells, typically human CD3 on human T cells, in particular human CD3 epsilon (CD3E). Anti-CD3 antibodies include, without limitation, teplizumab, otelixizumab, and visilizumab. Another non-limiting example of a suitable anti-CD3 antibody is OKT3, also known as muromonab, the UHCTI clone, also known as T3 and CD3E. OKT3 is murine anti-CD3 antibody (DrugBank Accession Number DB00075, which is hereby incorporated by reference in its entirety); Abz287a is humanized version of OKT3, Abz494 to Abz498 are pH-dependent antibodies. The sequence of Abz287a is found in GenBank Accession No. ALJ79286 and described in Pegu et al., “Activation and Lysis of Human CD4 Cells Latently Infected with HIV-1,” Nat. Commun. 6:8447 (2015), which are hereby incorporated by reference in their entirety.

Additional suitable immunomodulatory monoclonal antibodies include, without limitation, an anti-TNF-alpha antibody (e.g., infliximab, etanercept, adalimumab, golimumab, certolizumab pegol), an anti-IL1 antibody (e.g., canakinumab), an anti-CTLA-4 antibody (abatacept), an anti-thymocyte globulin antibody (e.g., antithymocyte globulin), an anti-CD6 antibody (e.g., itolizumab), an anti-CD20 antibody (e.g., rituximab), an anti-interleukin-21 antibody. See, e.g., Li et al., “Drugs for Autoimmune Inflammatory Diseases: From Small Molecule Compounds to Anti-TNF Biologics,” Front. Pharmacol. 8:460 (2017); von Herrath et al., “Anti-Interleukin-21 Antibody and Liraglutide for the Preservation of β-Cell function in Adults with Recent-Onset Type 1 Diabetes: A Randomised, Double-Blind, Placebo-Controlled, Phase 2 Trial,” Lancet Diabetes Endrocrinol. 9:212-224 (2021), and International Publication No. WO 2014/004857, which are hereby incorporated by reference in their entirety).

The identification of suitable immunomodulatory monoclonal antibody versions and fragments (e.g., anti-CD3 antibody versions and fragments) useful in carrying out methods of the present disclosure may be achieved by well-established and known methods and techniques in the art, such as by histidine substitution via phage display libraries or from combinatorial histidine substitution libraries by yeast surface display.

As used herein, the term “antibody” or “immunoglobulin” is used in the broadest sense and specifically covers intact monoclonal antibodies, polyclonal antibodies, multispecific antibodies (e.g., bispecific antibodies) formed from at least two intact antibodies, and antibody fragments, so long as they exhibit the desired biological activity. Depending on the amino acid sequence of their constant regions, intact or whole antibodies can be assigned to different classes. There are five major classes of intact antibodies: IgA, IgD, IgE, IgG, and IgM, and several of these may be further divided into “subclasses” (isotypes), e.g., IgG1, IgG2, IgG3, IgG4, IgA, and IgA2.

“Antibody fragments” according to the present disclosure comprise a portion of an intact antibody, such as comprising the antigen-binding or variable region thereof. Examples of antibody fragments include Fab, Fab′, F(ab′)2, Fv, single chain Fv (scFV) and Fc fragments, diabodies, linear antibodies, single-chain antibody molecules; bispecific and multispecific antibodies formed from antibody fragment(s).

In some embodiments, monovalent antibody fragments for antibodies according to the present disclosure are ScFV or Fab.

A “whole” or “complete” antibody according to the present disclosure is an antibody which comprises an antigen-binding variable region as well as a light chain constant domain (CL) and heavy chain constant domains, CH1, CH2, and CH3.

A “Fc” region of an antibody according to the present disclosure comprises, as a rule, a CH2, CH3 and the hinge region of an IgG1 or IgG2 antibody major class. The hinge region is a group of about 15 amino acid residues which combine the CH1 region with the CH2-CH3 region.

A “Fab” fragment also contains the constant domain of the light chain and the first constant domain (CH1) of the heavy chain and has one antigen-binding site only.

“Fab” fragments differ from Fab fragments by the addition of a few residues at the carboxy-terminus of the heavy chain CH1 domain including one or more cysteine residues from the antibody hinge region.

A “F(ab′)2” antibody according to the present disclosure is produced as pairs of Fab′ fragments which have hinge cysteines between them.

“Single-chain FV” or “scFv” antibody fragments according to the present disclosure comprise the VH and VL, domains of an antibody, where these domains are present in a single polypeptide chain. In some embodiments, the Fv polypeptide further comprises a polypeptide linker between the VH and VL domains which enables the scFv to form the desired structure for antigen binding.

The “variable domain” of an antibody according to the present disclosure comprises the framework regions (usually FR1 to FR4) as well as the CDR domains (usually CDR1, CDR2 and CDR3) which are designated as “hypervariable regions.”

The term “hypervariable region” or “CDR” when used herein refers to the amino acid residues of an antibody that are responsible for antigen-binding. The hypervariable region generally comprises amino acid residues from a “complementarity determining region” or “CDR” (e.g., residues 24-34 (L1), 50-56 (L2) and 89-97 (L3) in the light chain variable domain and 31-35 (H1), 50-65 (H2) and 95-102 (H3) in the heavy chain variable domain).

If not otherwise pointed out, the amino acid positions within the antibody molecules according to the present disclosure are numbered according to Kabat.

“Framework Region” or “FR” residues are those variable domain residues other than the hypervariable region residues as herein defined.

“Antibody variants” according to the present disclosure include antibodies that have a modified amino acid sequence compared to the parental antibody but have the same or changed binding affinity to the targeted antigen. Antibody variants differ from the parental antibody by replacement or deletion or addition of one or more amino acid residues at specific positions within the variable domains, including the CDR domains, and/or the constant regions of the antibody, in order to modify certain properties of the antibody, such as binding affinity and/or receptor functions, e.g., ADCC, FcRn binding, and the like. The histidine-mutated antibodies of the present disclosure without further modifications are not designated as “antibody variants” according to the present disclosure. Antibody variants according to the present disclosure exhibit a sequence homology of 80-99% compared to the parental antibody, or in some embodiments 90%, 91%, 92%, 93%, 94%, 95%, 96%, 97%, 98% and 99%, dependent on the specific location of the amino acid residue to be replaced, deleted or added.

The term “cytokine” is a generic term for proteins released by one cell population, which act on another cell as intercellular mediators. Examples of such cytokines are lymphokines, monokines, and traditional polypeptide hormones, such as vascular endothelial growth factor (VEGF); integrins thrombopoietin (TPO); nerve growth factors such as NGF.beta; platelet-growth factor; transforming growth factors (TGFs) such as TGFα and TGFβ; erythropoietin (EPO); interferons such as IFNα, IFNβ, and IFNγ; colony stimulating factors such as M-CSF, GM-CSF and G-CSF; interleukins such as IL-1, IL-la, IL-2, IL-3, IL-4, IL-5, IL-6, IL-7, IL-8, IL-9, IL-10, IL-11, IL-12, and TNF-α or TNF-β.

The term “humanized antibody” as used herein refers to a genetically engineered non-human antibody, which contains human antibody constant domains and non-human variable domains modified to contain a high level of sequence homology to human variable domains. This can be achieved by grafting of the six non-human antibody CDRs, which together form the antigen binding site, onto a homologous human acceptor framework region (FR) (see PCT Publication No. WO 92/22653 and European Patent No. 0629240, which are hereby incorporated by reference in their entirety). To fully reconstitute the binding affinity and specificity of the parental antibody, the substitution of framework residues from the parental antibody (i.e., the non-human antibody) into the human framework regions (back-mutations) may be required. Structural homology modeling may help to identify the amino acid residues in the framework regions that are important for the binding properties of the antibody. Thus, a humanized antibody may comprise non-human CDR sequences, primarily human framework regions optionally comprising one or more amino acid back-mutations to the non-human amino acid sequence, and fully human constant regions.

The term “human antibody” as used herein refers to antibodies having variable and constant regions derived from human germline immunoglobulin sequences. Human antibodies may include amino acid residues not encoded by human germline immunoglobulin sequences (e.g., mutations introduced by random or site-specific mutagenesis in vitro or by somatic mutation in vivo). However, the term “human antibody,” as used herein, is not intended to include antibodies in which CDR sequences derived from the germline of another mammalian species, such as a mouse, have been grafted onto human framework sequences. Human monoclonal antibodies of the present disclosure can be produced by a variety of techniques, including conventional monoclonal antibody methodology, e.g., the standard somatic cell hybridization technique of Kohler and Milstein, “Continuous Cultures of Fused Cells Secreting Antibody of Predefined Specificity,” Nature 256: 495 (1975), which is hereby incorporated by reference in its entirety. Although somatic cell hybridization procedures may be used, in principle, other techniques for producing monoclonal antibody can be employed, e.g., viral or oncogenic transformation of B-lymphocytes or phage display techniques using libraries of human antibody genes. A suitable animal system for preparing hybridomas that secrete human monoclonal antibodies is the murine system. Hybridoma production in the mouse is a very well-established procedure. Immunization protocols and techniques for isolation of immunized splenocytes for fusion are known in the art. Fusion partners (e.g., murine myeloma cells) and fusion procedures are also known. Human monoclonal antibodies can thus be generated using, e.g., transgenic or transchromosomal mice or rats carrying parts of the human immune system rather than the mouse or rat system. Accordingly, in some embodiments, a human antibody is obtained from a transgenic animal, such as a mouse or a rat, carrying human germline immunoglobulin sequences instead of animal immunoglobulin sequences. In such embodiments, the antibody originates from human germline immunoglobulin sequences introduced in the animal, but the final antibody sequence is the result of said human germline immunoglobulin sequences being further modified by somatic hypermutations and affinity maturation by the endogenous animal antibody machinery (see, e.g., Mendez et al., “Functional Transplant of Megabase Human Immunoglobulin Loci Recapitulates Human Antibody Response in Mice,” Nat. Genet. 15:146-56 (1997), which is hereby incorporated by reference in its entirety).

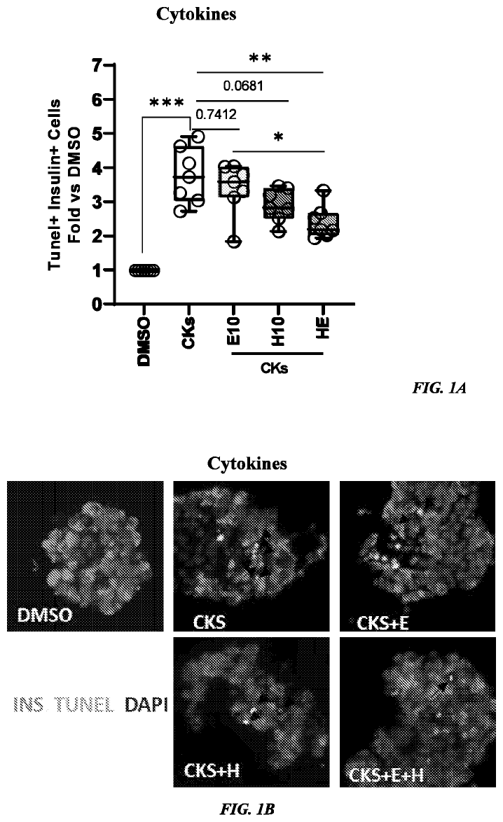

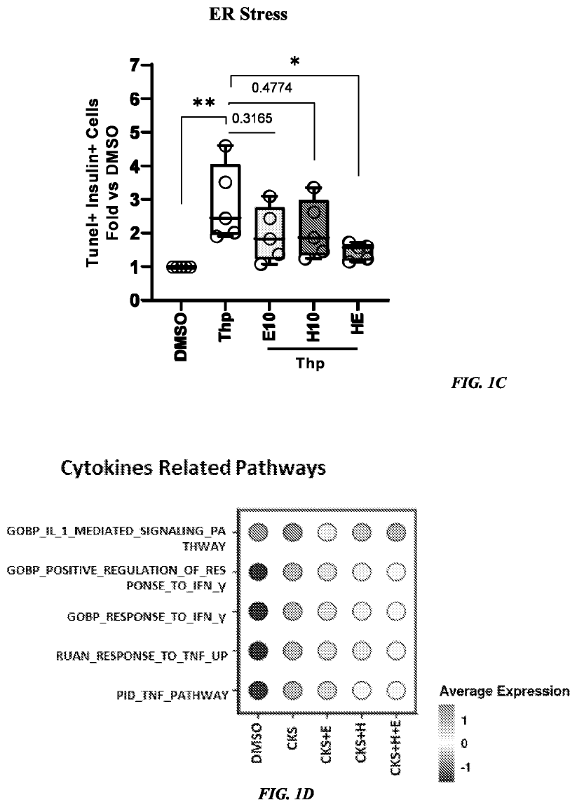

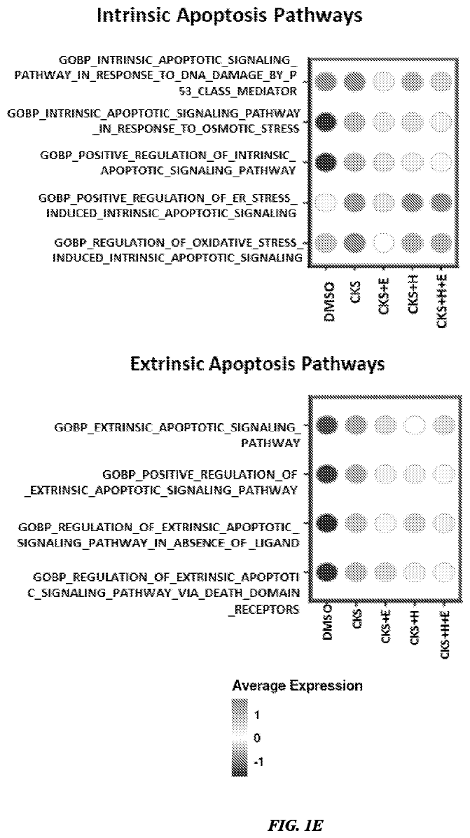

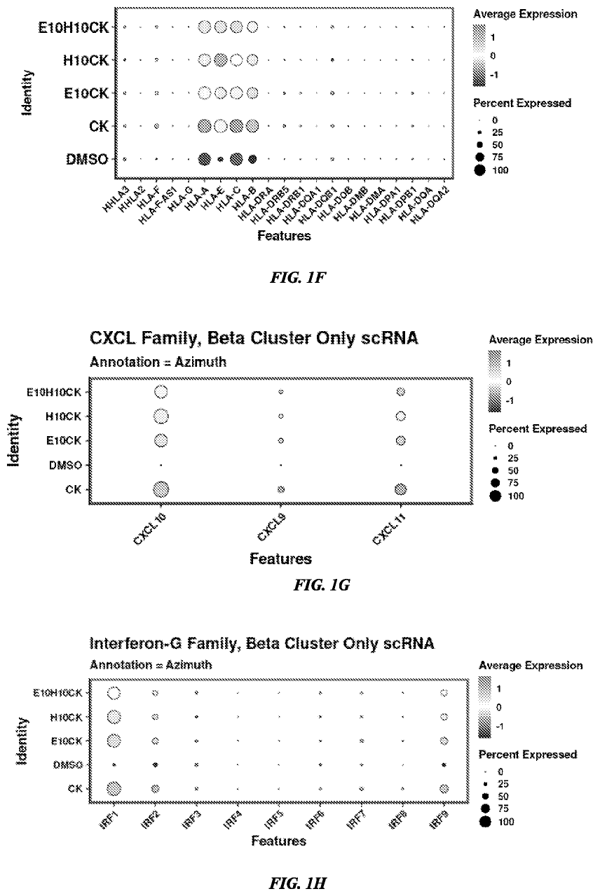

In some embodiments, the immunomodulatory monoclonal antibody (e.g., an anti-CD3 antibody) is administered at a low dose. As used herein, a “low dose” in reference to administering an immunomodulatory monoclonal antibody (e.g., an anti-CD3 antibody) is a suboptimal dose, or at a dose lower than would be administered if the immunomodulatory monoclonal antibody (e.g., an anti-CD3 antibody) were being administered as a treatment to a subject for a condition associated with insufficient insulin secretion on its own (i.e., without any other companion agents). In other words, an immunomodulatory monoclonal antibody (e.g., anti-CD3 antibody) administered pursuant to the methods described herein may be administered at a lower dose than usually administered for treatment of T1D due to the effects of the accompanying administering of the dual-specificity tyrosine phosphorylation-regulated kinase 1A (DYRK1A) inhibitor and the glucagon-like peptide-1 receptor (GLP1R) agonist. In Bresson et al., “Anti-CD3 and Nasal Proinsulin Combination Therapy Enhances Remission from Recent-Onset Autoimmune Diabetes by Inducing Tregs,” J. Clin. Invest. 116(5):1371-1381 (2006) (“Bresson”), which is hereby incorporated by reference in its entirety, an anti-CD3 antibody was tested at different doses for its capacity to reverse T1D in NOD mice (see Bresson, FIG. 1E). Bresson chose 40 μg/day as a “suboptimal” dose that leads to reversal of diabetes in only 30% of the mice since their goal was to test whether combination with other drugs (in that case nasal insulin) could further improve the reversal of diabetes in these mice. In the examples discussed in the present disclosure below, a “suboptimal” dose of anti-CD3 was used and its combination with harmine plus exenatide (a 39 amino acid peptide that is the synthetic version of Exendin-4) treatment reversed diabetes in 100% of the diabetic NOD mice. In some embodiments, the suboptimal dose of an anti-CD3 antibody for a human patient is less than the dose provided in a 14-day course of escalating doses of intravenous teplizumab, with a total cumulative dose of about 9034 mg/m²), and further defined as (day 1, 51 mg/m²; day 2, 103 mg/m²; day 3, 206 mg/m²; day 4, 413 mg/m²; days 5*C14, 826 mg/m²; median cumulative dose 11.6 mg; interquartile range 5.7 mg). Thus, in some embodiments, the dose of the anti-CD3 antibody is less than ½ or less than ¼ of the dose provided in a 14-day course of escalating doses of intravenous teplizumab.

Also contemplated are methods of treating a subject for a condition associated with insufficient insulin secretion. This method involves administering to a subject in need of treatment for a condition associated with an insufficient level of insulin secretion a dual-specificity tyrosine phosphorylation-regulated kinase 1A (DYRK1A) inhibitor, a glucagon-like peptide-1 receptor (GLP1R) agonist, and an immunosuppressive agent, where said administering is carried out under conditions effective to reverse loss of β-cell mass and function in the subject to treat the subject for the condition associate with insufficient insulin secretion.

Suitable DYRK1A inhibitors and GLP1R agonists are described in detail supra.

Suitable immunosuppressive agents include, without limitation, immunomodulatory monoclonal antibodies (e.g., an anti-CD3 antibody), as well as tacrolimus, rapamycin, mycophenylate mofetil, and glucocorticoids such as prednisone, cortisone, and dexamethasone.

As used herein, a condition associated with an insufficient level of insulin secretion means a condition where a subject produces a lower plasma level of insulin than is required to maintain normal glucose levels in the blood such that the subject with the condition associated with insufficient insulin secretion becomes hyperglycemic. In such a condition, the pancreatic beta cells of the afflicted subject secrete an insufficient level of insulin to maintain the presence of a normal concentration of glucose in the blood (i.e., normoglycemica).

One of the conditions associated with an insufficient level of insulin secretion is insulin resistance. Insulin resistance is a condition in which a subject's cells become less sensitive to the glucose-lowering effects of insulin. Insulin resistance in muscle and fat cells reduces glucose uptake (and, therefore, local storage of glucose as glycogen and triglycerides), whereas insulin resistance in liver cells results in reduced glycogen synthesis and storage and a failure to suppress glucose production and release into the blood. Insulin resistance normally refers to reduced glucose-lowering effects of insulin. However, other functions of insulin can also be affected. For example, insulin resistance in fat cells reduces the normal effects of insulin on lipids and results in reduced uptake of circulating lipids and increased hydrolysis of stored triglycerides. Increased mobilization of stored lipids in these cells elevates free fatty acids in the blood plasma. Elevated blood fatty-acid concentrations, reduced muscle glucose uptake, and increased liver glucose production all contribute to elevated blood glucose levels. If insulin resistance exists, more insulin needs to be secreted by the pancreas. If this compensatory increase does not occur, blood glucose concentrations increase and type II diabetes occurs.

One of the conditions associated with an insufficient level of insulin secretion is diabetes. Diabetes can be divided into two broad types of diseases: Type I (“T1D”) and Type II (“T2D”).

In some embodiments, the condition associated with an insufficient level of insulin secretion is type 1A diabetes or immune-mediated diabetes. In some embodiments, the condition associated with an insufficient level of insulin secretion is type 1B diabetes or idiopathic diabetes.

The term “diabetes” also refers herein to a group of metabolic diseases in which patients have high blood glucose levels, including Type I diabetes, Type II diabetes, gestational diabetes, congenital diabetes, maturity onset diabetes (“MODY”), cystic fibrosis-related diabetes, hemochromatosis-related diabetes, drug-induced diabetes (e.g., steroid diabetes), and several forms of monogenic diabetes.

In certain embodiments, the subject has or is being treated for one or more of Type I diabetes (T1D), Type IA diabetes, Type 1B diabetes, Type II diabetes (T2D), gestational diabetes, congenital diabetes, maturity onset diabetes (MODY), cystic fibrosis-related diabetes, hemochromatosis-related diabetes, drug-induced diabetes, or monogenic diabetes. For example, the subject has or is being treated for Type I diabetes. Or, the subject has or is being treated for Type II diabetes.

In some embodiments, the subject as long term T1D. In some embodiments the subject has recent onset T1D. See, e.g., Coppieters et al., “Demonstration of Islet-Autoreactive CD8 T Cells in Insulitic Lesions from Recent Onset and Long-Term Type 1 Diabetes Patients,” J. Exp. Med. 209:51-60 (2012), which is hereby incorporated by reference in its entirety, for descriptions of long term T1D and recent onset T1D.

In some embodiments, the subject has a disease or disorder associated with mutant and/or aberrant expression or function of DYRK1A. In accordance with such embodiments, the subject may have Down's syndrome. Down syndrome is associated with an increased incidence of autoimmune diseases such as an increased risk and prevalence of type 1 diabetes.

Treatment methods described herein are effective to treat a subject with an insufficient level of insulin secretion by, for example, increasing immune tolerance in the subject, enhancing β-cell proliferation in the subject, protecting β-cells in the subject, increasing β-cells mass in the subject, or any combination thereof.

In some embodiments, the condition associated with an insufficient level of insulin secretion is metabolic syndrome. Metabolic syndrome is generally used to define a constellation of abnormalities that is associated with increased risk for the development of type II diabetes and atherosclerotic vascular disease. Related conditions and symptoms include, but are not limited to, fasting hyperglycemia (diabetes mellitus type II or impaired fasting glucose, impaired glucose tolerance, or insulin resistance); high blood pressure; central obesity (also known as visceral, male-pattern or apple-shaped adiposity), meaning overweight with fat deposits mainly around the waist; decreased HDL cholesterol; and elevated triglycerides.

In some embodiments, the condition associated with an insufficient level of insulin secretion is metabolic syndrome or insulin resistance, and methods described herein are carried out to treat a subject having or being treated for metabolic syndrome or insulin resistance.

Other conditions that may be associated with an insufficient level of insulin secretion include, without limitation, hyperuricemia, fatty liver (especially in concurrent obesity) progressing to non-alcoholic fatty liver disease, polycystic ovarian syndrome (in women), and acanthosis nigricans.

Related disorders may also be treated pursuant to the treatment methods disclosed herein including, without limitation, any disease associated with a blood or plasma glucose level outside the normal range, such as hyperglycemia. Consequently, the term “related disorders” includes impaired glucose tolerance (“IGT”), impaired fasting glucose (“IFG”), insulin resistance, metabolic syndrome, postprandial hyperglycemia, and overweight/obesity. Such related disorders can also be characterized by an abnormal blood and/or plasma insulin level.

Methods described herein may be carried out to treat a subject with conditions associated with beta cell failure or deficiency. Such conditions include, without limitation, Type I diabetes (T1D), Type II diabetes (T2D), gestational diabetes, congenital diabetes, maturity onset diabetes (MODY), cystic fibrosis-related diabetes, hemochromatosis-related diabetes, drug-induced diabetes, or monogenic diabetes. Drug induced diabetes relates to a condition that is caused through the use of drugs that are toxic to beta cells (e.g., steroids, antidepressants, second generation antipsychotics, and immunosuppressives). Exemplary immunosuppressive drugs include, but are not limited to, members of the cortisone family (e.g., prednisone and dexamethasome), rapamycin/sirolimus, everolimus, and calciuneurin inhibitors (e.g., FK-506/tacrolimus).

Other conditions associated with beta cell deficiency include, without limitation, hypoglycemia unawareness, labile insulin dependent diabetes, pancreatectomy, partial pancreatectomy, pancreas transplantation, pancreatic islet allotransplantation, pancreatic islet autotransplantation, and pancreatic islet xenotransplantation.

As used herein, hypoglycemia unawareness is a complication of diabetes in which the patient is unaware of a deep drop in blood sugar because it fails to trigger the secretion of epinephrine which generates the characteristic symptoms of hyperglycemia (e.g., palpitations, sweating, anxiety) that serve to warn the patient of the dropping blood glucose.

Pancreas transplantation may occur alone, after, or in combination with kidney transplantation. For example, pancreas transplantation alone may be considered medically necessary in patients with severely disabling and potentially life-threatening complications due to hypoglycemia unawareness and labile insulin dependent diabetes that persists in spite of optimal medical management. Pancreas transplantation following prior kidney transplantation may occur in a patient with insulin dependent diabetes. Pancreas transplantation may occur in combination with kidney transplantation in an insulin dependent diabetic patient with uremia. Pancreas retransplantation may be considered after a failed primary pancreas transplant.

As used herein, pancreatic islet transplantation is a procedure in which only the islets of Langerhans, which contain the endocrine cells of the pancreas, including the insulin producing beta cells and glucagon producing alpha cells, are isolated and transplanted into a patient. Pancreatic islet allotransplantation occurs when islets of Langerhans are isolated from one or more human donor pancreas. Pancreatic islet cells may also be derived from human embryonic stem cells or induced pluripotent stem cells. Pancreatic islet xenotransplantation occurs when islets of Langerhans are isolated from one or more non-human pancreas (e.g., a porcine pancreas or primate pancreas). Pancreatic islet autotransplantation occurs when islets of Langerhans are isolated from the pancreas of a patient undergoing pancreatectomy (e.g., for chronic pancreatitis from gall stone, drugs, and/or familial genetic causes) and returned to the same patient via infusion into the portal vein, via laparoscopy to the omentum, via endoscopy to the gastric wall, or subcutaneously via minor incision. As with pancreas transplantation, pancreatic islet transplantation can be performed alone, after, or in combination with kidney transplantation. For example, pancreatic islet transplantation may occur alone to restore hypoglycemia awareness, provide glycemic control, and/or protect a patient from severe hypoglycemic events (Hering et al., “Phase 3 Trial of Transplantation of Human Islets in Type 1 Diabetes Complicated by Severe Hypoglycemia,” Diabetes Care 39(7):1230-1240 (2016), which is hereby incorporated by reference in its entirety).

Pancreatic islet transplantation may occur in combination with total pancreatectomy. For example, pancreatic islet transplantation may be performed after total pancreatectomy to prevent or ameliorate surgically induced diabetes by preserving R cell function (Johnston et al., “Factors Associated With Islet Yield and Insulin Independence After Total Pancreatectomy and Islet Cell Autotransplantation in Patients With Chronic Pancreatitis Utilizing Off-site Islet Isolation: Cleveland Clinic Experience,” J. Chem. Endocrinol. Metab. 100(5):1765-1770 (2015), which is hereby incorporated by reference in its entirety). Thus, pancreatic islet transplantation may provide sustained long-term insulin-independence.

In some embodiments, pancreatic islet transplantation may occur in combination with the administration of immunosuppressive agents. Suitable immunosuppressive agents include, but are not limited to, daclizumab (Zenapax; Roche), low-dose rapamycin (sirolimus), and FK506 (tacrolimus) (Van Belle et al., “Immunosuppression in Islet Transplantation,” J. Clin. Invest. 118(5):1625-1628 (2008), which is hereby incorporated by reference in its entirety).

In some embodiments, pancreatic islet transplantation occurs in the context of an encapsulation device to protect the transplanted pancreatic islet cells from the host autoimmune response, while allowing glucose and nutrients to reach the transplanted pancreatic islet cells.

The methods described herein may be carried out to enhance pancreas, pancreatic islet allotransplantation, pancreatic islet autotransplantation, pancreatic islet xenotransplantation by regenerating pancreatic β cells in a patient. For example, the methods of the present disclosure may be used to prevent or ameliorate surgically induced diabetes by preserving p cell function, restore hypoglycemia awareness, provide glycemic control, and/or protect a patient from severe hypoglycemic events. Thus, other aspects of the present disclosure relate to methods of regenerating pancreatic beta cells in a transplant patient. Such methods involve administering to a transplant patient a dual-specificity tyrosine phosphorylation-regulated kinase 1A (DYRK1A) inhibitor, a glucagon-like peptide-1 receptor (GLP1R) agonist, and an anti-CD3 antibody, where said administering is carried out under conditions effective to reverse loss of 3-cell mass and function in the subject to treat the transplant patient.

The methods may be carried out to treat a subject at risk of developing Type II Diabetes. A patient at risk of developing Type II Diabetes may have pre-diabetes/metabolic syndrome.

A patient at risk of developing Type II Diabetes may have been treated with a psychoactive drug including, but not limited to, a selective serotonin reuptake inhibitor (“SSRI”) for depression, obsessive compulsive disorder (“OCD”), etc.

The subject may be a mammalian subject, for example, a human subject. Suitable human subjects include, without limitation, children, adults, and elderly subjects having a beta-cell and/or insulin deficiency.

The subject may also be non-human, such as bovine, ovine, porcine, feline, equine, murine, canine, lapine, etc.

Administering to a subject a dual-specificity tyrosine phosphorylation-regulated kinase 1A (DYRK1A) inhibitor, a glucagon-like peptide-1 receptor (GLP1R) agonist, and an immunomodulatory monoclonal antibody and/or an immunosuppressive agent (e.g., an anti-CD3) antibody may increase the number of proliferating pancreatic beta cells in the subject by at least about 4%, 5%, 6%, 7%, 8%, 9%, 10%, or more.

Administering to a subject a dual-specificity tyrosine phosphorylation-regulated kinase 1A (DYRK1A) inhibitor, a glucagon-like peptide-1 receptor (GLP1R) agonist, and an immunomodulatory monoclonal antibody and/or an immunosuppressive agent (e.g., anti-CD3 antibody) may increase the number of proliferating pancreatic beta cells in a subject by about 4-10% per day, or about 4-6% per day, 5-7% per day, 6-9% per day, or 7-10% per day.

Administering to a subject a dual-specificity tyrosine phosphorylation-regulated kinase 1A (DYRK1A) inhibitor, a glucagon-like peptide-1 receptor (GLP1R) agonist, and an immunomodulatory monoclonal antibody and/or an immunosuppressive agent (e.g., anti-CD3 antibody) may increase the number of proliferating pancreatic beta cells in the subject by about 6-10% per day.

Administering to a subject a dual-specificity tyrosine phosphorylation-regulated kinase 1A (DYRK1A) inhibitor, a glucagon-like peptide-1 receptor (GLP1R) agonist, and an immunomodulatory monoclonal antibody and/or an immunosuppressive agent (e.g., anti-CD3 antibody) may increase glucose-stimulated insulin secretion in pancreatic beta cells of the subject (e.g., compared to a subject not administered a dual-specificity tyrosine phosphorylation-regulated kinase 1A (DYRK1A) inhibitor, a glucagon-like peptide-1 receptor (GLP1R) agonist, and an immunomodulatory monoclonal antibody and/or an immunosuppressive agent (e.g., anti-CD3 antibody)).

Administering to a subject a dual-specificity tyrosine phosphorylation-regulated kinase 1A (DYRK1A) inhibitor, a glucagon-like peptide-1 receptor (GLP1R) agonist, and an anti-CD3 antibody may be carried out by administering a single composition comprising all three of the DYRK1A inhibitor, the GLP1R agonist, and the immunomodulatory monoclonal antibody and/or an immunosuppressive agent (e.g., the anti-CD3 antibody). Alternatively, administering to a subject a dual-specificity tyrosine phosphorylation-regulated kinase 1A (DYRK1A) inhibitor, a glucagon-like peptide-1 receptor (GLP1R) agonist, and an immunomodulatory monoclonal antibody and/or an immunosuppressive agent (e.g., an anti-CD3 antibody) may be carried out serially. For example, a subject may first be administered an immunomodulatory monoclonal antibody and/or an immunosuppressive agent (e.g., an anti-CD3 antibody) and then a dual-specificity tyrosine phosphorylation-regulated kinase 1A (DYRK1A) inhibitor (or a compositions comprising the dual-specificity tyrosine phosphorylation-regulated kinase 1A (DYRK1A) inhibitor) and then a glucagon-like peptide-1 receptor (GLP1R) agonist (or a compositions comprising the glucagon-like peptide-1 receptor (GLP1R) agonist) or, after being administered an immunomodulatory monoclonal antibody and/or an immunosuppressive agent (e.g., an anti-CD3 antibody), then administered a glucagon-like peptide-1 receptor (GLP1R) agonist (or composition thereof) and then a dual-specificity tyrosine phosphorylation-regulated kinase 1A (DYRK1A) inhibitor (or composition thereof) or, in yet another embodiment, after being administered an immunomodulatory monoclonal antibody and/or an immunosuppressive agent (e.g., an anti-CD3 antibody) then being administered a combination therapy of a dual-specificity tyrosine phosphorylation-regulated kinase 1A (DYRK1A) inhibitor (or a compositions comprising the dual-specificity tyrosine phosphorylation-regulated kinase 1A (DYRK1A) inhibitor) and a glucagon-like peptide-1 receptor (GLP1R) agonist (or a compositions comprising the glucagon-like peptide-1 receptor (GLP1R) agonist).

Administering of any one or a combination of a dual-specificity tyrosine phosphorylation-regulated kinase 1A (DYRK1A) inhibitor, a glucagon-like peptide-1 receptor (GLP1R) agonist, and an immunomodulatory monoclonal antibody and/or an immunosuppressive agent (e.g., anti-CD3 antibody) may occur multiple times each day, daily, weekly, twice weekly, monthly, bi-monthly, annually, semi-annually, or any amount of time there between. The immunomodulatory monoclonal antibody and/or an immunosuppressive agent (e.g., the anti-CD3 antibody), DYRK1A inhibitor, and the glucagon-like peptide-1 receptor (GLP1R) agonist may be administered at the same or different administration frequencies. In some embodiments, administering of any one or more of a dual-specificity tyrosine phosphorylation-regulated kinase 1A (DYRK1A) inhibitor, a glucagon-like peptide-1 receptor (GLP1R) agonist, and an immunomodulatory monoclonal antibody and/or an immunosuppressive agent (e.g., an anti-CD3 antibody) is carried out acutely or chronically. For example, administering may be carried out chronically over a period of 1 year, 2 years, 3, years, 4 years, or more. In some embodiments, administering is carried out infrequently.

As used herein, the term “treating” is meant preventive or improved or curative treatment. In other words, treatment methods may be carried out to prevent a subject from getting a condition associated with insufficient insulin secretion or from a subject's condition associated with insufficient insulin secretion getting worse. Alternatively, the treatment method is carried out to improve a subject's condition associated with insufficient insulin secretion, or to fully cure the condition (i.e., such that the subject no longer has a condition associated with an insufficient level of insulin secretion as judged by a competent health care professional).

In some embodiments, “treating” is carried out to reverse loss of β-cell mass and function in a subject with T1D. In some embodiments, “treating” is carried out to prevent progression of T1D in a subject with recent onset T1D. In other words, methods of the present disclosure can be carried out to restore loss of β-cell mass and function in a subject that has lost β-cell mass and function due to a condition associated with insufficient insulin secretion, such as from T1D. In some embodiments, “treating” is carried out to prevent progression of T1D in a subject at risk of developing T1D.

The term “treating” means the correction, decrease in the rate of change, or reduction of an impaired glucose homeostasis in a subject. The level of glucose in blood fluctuates throughout the day. Glucose levels are usually lower in the morning, before the first meal of the day and rise after meals for some hours. Consequently, the term “treating” includes controlling blood glucose level in a subject by increasing or decreasing the subject's blood glucose level. This may depend on many factors, including the condition of the subject and/or the particular time of day, as blood glucose levels fluctuate throughout the day.

“Treating” means regulating a temporary or persistent reduction of blood glucose level in a subject having diabetes or a related disorder. The term “treating” may also mean improving insulin release (e.g., by pancreatic beta cells) in a subject.

It may be desirable to modulate blood glucose levels in a subject to normalize or regulate the blood or plasma glucose level in a subject having abnormal levels (i.e., levels that are below or above a known reference, median, or average value for a corresponding subject with a normal glucose homeostasis). The treatment methods of the present disclosure may be carried out to achieve such effects.

In carrying out treatment methods of the present disclosure, administering of a dual-specificity tyrosine phosphorylation-regulated kinase 1A (DYRK1A) inhibitor, a glucagon-like peptide-1 receptor (GLP1R) agonist, and an immunomodulatory monoclonal antibody and/or an immunosuppressive agent (e.g., anti-CD3 antibody) to a subject may involve administering a pharmaceutical composition comprising the dual-specificity tyrosine phosphorylation-regulated kinase 1A (DYRK1A) inhibitor or the glucagon-like peptide-1 receptor (GLP1R) agonist or an immunomodulatory monoclonal antibody and/or an immunosuppressive agent (e.g., anti-CD3 antibody), or all three, in therapeutically effective amounts, which means an amount of the DYRK1A inhibitor, the GLP1R agonist, and the immunomodulatory monoclonal antibody and/or the immunosuppressive agent (e.g., anti-CD3 antibody) effective to treat the stated conditions and/or disorders in the subject. Such amounts generally vary according to a number of factors well within the purview of a person of ordinary skill in the art. These include, without limitation, the particular subject's general health, age, weight, height, general physical condition, medical history, the particular compound used, as well as the carrier in which it is formulated, and the route of administration selected for it, the length or duration of treatment, and the nature and severity of the condition being treated.

Administering typically involves administering pharmaceutically acceptable dosage forms, which means dosage forms of compounds described herein and includes, for example, tablets, dragees, powders, elixirs, syrups, liquid preparations, including suspensions, sprays, inhalants tablets, lozenges, emulsions, solutions, granules, capsules, and suppositories, as well as liquid preparations for injections, including liposome preparations. Techniques and formulations generally may be found in Remington 's Pharmaceutical Sciences, Mack Publishing Co., Easton, Pa., latest edition, which is hereby incorporated by reference in its entirety.

In carrying out treatment methods, the DYRK1A inhibitor, the GLP1R agonist, and/or the anti-CD3 antibody may be contained, in any appropriate amount, in any suitable carrier substance. DYRK1A inhibitors, the GLP1R agonists, and an anti-CD3 antibody may be present in an amount of up to 99% by weight of the total weight of the composition. The composition may be provided in a dosage form that is suitable for the oral, parenteral (e.g., intravenously, intramuscularly), rectal, cutaneous, nasal, vaginal, inhalant, skin (patch), or ocular administration route. Thus, the composition may be in the form of, e.g., tablets, capsules, pills, powders, granulates, suspensions, emulsions, solutions, gels including hydrogels, pastes, ointments, creams, plasters, drenches, osmotic delivery devices, suppositories, enemas, injectables, implants, sprays, or aerosols.

Pharmaceutical compositions may be formulated to release the active DYRK1A inhibitor, the GLP1R agonist, and the immunomodulatory monoclonal antibody and/or an immunosuppressive agent (e.g., anti-CD3 antibody) substantially immediately upon administration or at any predetermined time or time period after administration.

Controlled release formulations include (i) formulations that create a substantially constant concentration of the drug(s) within the body over an extended period of time; (ii) formulations that after a predetermined lag time create a substantially constant concentration of the drug(s) within the body over an extended period of time; (iii) formulations that sustain drug(s) action during a predetermined time period by maintaining a relatively, constant, effective drug level in the body with concomitant minimization of undesirable side effects associated with fluctuations in the plasma level of the active drug substance; (iv) formulations that localize drug(s) action by, e.g., spatial placement of a controlled release composition adjacent to or in the diseased tissue or organ; and (v) formulations that target drug(s) action by using carriers or chemical derivatives to deliver the drug to a particular target cell type.

Administration of DYRK1A inhibitor(s), GLP1R agonist(s), and immunomodulatory monoclonal antibodies and/or immunosuppressive agents (e.g., an anti-CD3 antibody) in the form of a controlled release formulation may be preferable in cases in which the drug has (i) a narrow therapeutic index (i.e., the difference between the plasma concentration leading to harmful side effects or toxic reactions and the plasma concentration leading to a therapeutic effect is small; in general, the therapeutic index (“TI”) is defined as the ratio of median lethal dose (LD₅₀) to median effective dose (ED₅₀)); (ii) a narrow absorption window in the gastro-intestinal tract; or (iii) a very short biological half-life so that frequent dosing during a day is required in order to sustain the plasma level at a therapeutic level.

DYRK1A inhibitor(s), GLP1R agonist(s), and immunomodulatory monoclonal antibodies and/or immunosuppressive agent(s) (e.g., an anti-CD3 antibody) can be used enterally or parenterally. Orally, the agents to be administered may be administered in the amount from about 0.1 mg per day to 1,000 mg per day. For parenteral, sublingual, intranasal, or intrathecal administration, the compounds according to the present disclosure may be used in an amount from about 0.5 to about 100 mg/day; for depo administration and implants from about 0.5 mg/day to about 50 mg/day; for topical administration from about 0.5 mg/day to about 200 mg/day; for rectal administration from about 0.5 mg to about 500 mg. In some embodiments, therapeutically effective amounts for oral administration is from about 1 mg/day to about 100 mg/day; and for parenteral administration from about 5 to about 50 mg daily. In some embodiments, the therapeutically effective amounts for oral administration are from about 5 mg/day to about 50 mg/day.

A daily dosage of active ingredient can be expected to be about 0.001 to about 1000 milligrams per kilogram of body weight, with the preferred dose being about 0.1 to about 30 mg/kg. The daily oral dosage can vary from about 0.01 mg to 1000 mg, 0.1 mg to 100 mg, or 10 mg to 500 mg per day of a compound. The daily dose may be administered as single dose or in divided doses and, in addition, the upper limit can also be exceeded when this is found to be indicated.

Any of a number of strategies can be pursued to obtain controlled release in which the rate of release outweighs the rate of metabolism of the therapeutic agents in question. Controlled release may be obtained by appropriate selection of various formulation parameters and ingredients, including, e.g., various types of controlled release compositions and coatings. Thus, the drug is formulated with appropriate excipients into a pharmaceutical composition that, upon administration, releases the drug in a controlled manner (single or multiple unit tablet or capsule compositions, oil solutions, suspensions, emulsions, microcapsules, microspheres, nanoparticles, patches, and liposomes). Thus, administering may be carried out nasally, orally, topically, transdermally, parenterally, subcutaneously, intravenously, intramuscularly, intraperitoneally, by intranasal instillation, by intracavitary or intravesical instillation, intraocularly, intraarterially, intralesionally, or by application to mucous membranes. Compounds may be administered alone or with suitable pharmaceutical carriers, and can be in solid or liquid form, such as tablets, capsules, powders, solutions, suspensions, or emulsions. In certain embodiments, administering is carried out nasally, orally, transdermally, parenterally, subcutaneously, intravenously, intramuscularly, or intraperitoneally.

In certain embodiments, the administering is carried out using an infusion pump to provide, e.g., rate controlled infusion, periodic infusion, and/or bolus dosage infusion. The infusion pump may be a stationary or ambulatory infusion pump. Stationary infusion pumps are used primarily at a patient's bedside. Ambulatory infusion pumps are relatively small, at least substantially self-contained devices that are used to introduce drugs and other infusible substances (e.g., insulin) to a selected subject. Some ambulatory infusion pumps are configured to be worn on a belt, carried in a clothing pocket, or otherwise supported within a holder of some kind (collectively referred to as “pocket pumps”). Other infusion pumps are configured to adhere to the skin in a patch-like fashion (referred to as “patch pumps”). Infusion pumps may be used, for example, to intravenously or subcutaneously introduce (or “infuse”) medicament on an ongoing or even continuous basis outside of a clinical environment. Infusion pumps greatly reduce the frequency of subcutaneous access events such as needle-based shots. In certain embodiments, the infusion pump is a subcutaneous or intravenous infusion pump. For example, the infusion pump may be an ambulatory subcutaneous insulin infusion pump.

Another aspect of the present disclosure relates to a composition comprising a dual-specificity tyrosine phosphorylation-regulated kinase 1A (DYRK1A) inhibitor, a glucagon-like peptide-1 receptor (GLP1R) agonist, and an immunomodulatory monoclonal antibody (e.g., an anti-CD3 antibody).

Suitable DYRK1A inhibitors are described supra and include, e.g., harmine, INDY, leucettine-41, 5-iodotubercidin (5-IT), GNF4877, CC-401, kinase inhibitors, and derivatives thereof.

Suitable GLP1R agonists are described supra and include, e.g., exendin-4, liraglutide, lixisenatide, semaglutide, and derivatives thereof.

Suitable immunomodulatory monoclonal antibodies are described in detail supra. Suitable anti-CD3 antibodies are described above and include, e.g., teplizumab.

Also contemplated are compositions comprising a dual-specificity tyrosine phosphorylation-regulated kinase 1A (DYRK1A) inhibitor, a glucagon-like peptide-1 receptor (GLP1R) agonist, and an immunosuppressive agent. Suitable immunosuppressive agents are described in detail supra.

The composition may further comprise a carrier. Suitable carriers are described supra. The carrier may be a pharmaceutically-acceptable carrier. Suitable pharmaceutically-acceptable carriers are described supra.

A further aspect of the present disclosure relates to a method of increasing β-cell mass and function in a population of pancreatic beta cells. This method involves contacting a population of pancreatic beta cells with a dual-specificity tyrosine phosphorylation-regulated kinase 1A (DYRK1A) inhibitor, a glucagon-like peptide-1 receptor (GLP1R) agonist, and a low dose of an anti-CD3 antibody, where said contacting is carried out under conditions effective to increase β-cell mass and function in the population of pancreatic beta cells.

In carrying out this and other methods described herein, the pancreatic beta cells may be mammalian cells. Mammalian cells include cells from, for example, mice, hamsters, rats, cows, sheep, pigs, goats, horses, monkeys, dogs (e.g., Canisfamiliaris), cats, rabbits, guinea pigs, and primates, including humans. For example, the cells may be human pancreatic beta cells.

In some embodiments, “pancreatic beta cells” are primary human pancreatic beta cells.

In some embodiments, this and other methods described herein are carried out ex vivo or in vivo. When carried out ex vivo, a population of cells may be provided by obtaining cells from a pancreas and culturing the cells in a liquid medium suitable for the in vitro or ex vivo culture of mammalian cells, in particular human cells. For example, and without limitation, a suitable and non-limiting culture medium may be based on a commercially available medium such as RPMI1640 from Invitrogen.

Methods for determining whether a cell has a pancreatic beta cell phenotype are known in the art and include, without limitation, incubating the cell with glucose and testing whether insulin expression in the cell is increased or induced. Other methods include testing whether beta cell specific transcription factors are expressed, the detection of beta cell specific gene products with the help of RNA quantitative PCR, the transplantation of a candidate cell in diabetic mice, and subsequent testing of the physiologic response following said transplantation as well as analyzing the cells with electron microscopy.

In carrying out methods described herein, a population of pancreatic beta cells is contacted with a dual-specificity tyrosine phosphorylation-regulated kinase 1A (DYRK1A) inhibitor, a glucagon-like peptide-1 receptor (GLP1R) agonist, and an anti-CD3 antibody.

In some embodiments, contacting a population of pancreatic beta cells with a dual-specificity tyrosine phosphorylation-regulated kinase 1A (DYRK1A) inhibitor, a glucagon-like peptide-1 receptor (GLP1R) agonist, and an anti-CD3 antibody is carried out with harmine, exendin-4, and teplizumab.

Contacting a population of pancreatic beta cells with a dual-specificity tyrosine phosphorylation-regulated kinase 1A (DYRK1A) inhibitor, a glucagon-like peptide-1 receptor (GLP1R) agonist, and an immunomodulatory monoclonal antibody and/or an immunosuppressive agent (e.g., an anti-CD3 antibody) may be carried out with a single composition comprising each of the DYRK1A inhibitor, the GLP1R agonist, and the immunomodulatory monoclonal antibody and/or the immunosuppressive agent (e.g., an anti-CD3 antibody). Alternatively, contacting a population of pancreatic beta cells with a dual-specificity tyrosine phosphorylation-regulated kinase 1A (DYRK1A) inhibitor, a glucagon-like peptide-1 receptor (GLP1R) agonist, and an immunomodulatory monoclonal antibody and/or an immunosuppressive agent (e.g., anti-CD3 antibody) may be carried out serially. For example, a population of pancreatic beta cells may first be contacted with an immunomodulatory monoclonal antibody and/or an immunosuppressive agent (e.g., anti-CD3 antibody) and then a dual-specificity tyrosine phosphorylation-regulated kinase 1A (DYRK1A) inhibitor (or a compositions comprising the dual-specificity tyrosine phosphorylation-regulated kinase 1A (DYRK1A) inhibitor) and a glucagon-like peptide-1 receptor (GLP1R) agonist (or a compositions comprising the glucagon-like peptide-1 receptor (GLP1R) agonist) (together or separately).

In carrying out methods described herein, contacting a population of pancreatic beta cells with a dual-specificity tyrosine phosphorylation-regulated kinase 1A (DYRK1A) inhibitor, a glucagon-like peptide-1 receptor (GLP1R) agonist, and an immunomodulatory monoclonal antibody and/or an immunosuppressive agent (e.g., anti-CD3 antibody) may occur multiple times a day, daily, weekly, twice weekly, monthly, bi-monthly, annually, semi-annually, or any amount of time there between. The DYRK1A inhibitor, the glucagon-like peptide-1 receptor (GLP1R) agonist, and the immunomodulatory monoclonal antibody and/or the immunosuppressive agent (e.g., anti-CD3 antibody) may be administered at different administration frequencies. Contacting a population of pancreatic beta cells with a DYRK1A inhibitor, a GLP1R, and an immunomodulatory monoclonal antibody and/or an immunosuppressive agent (e.g., anti-CD3 antibody) agonist may occur acutely or chronically. For example, contacting may occur chronically over a period of 1 year, 2 years, 3 years, 4 years, or more. In some embodiments, administering is carried out infrequently.

In some embodiments, contacting a population of pancreatic beta cells with a dual-specificity tyrosine phosphorylation-regulated kinase 1A (DYRK1A) inhibitor, a glucagon-like peptide-1 receptor (GLP1R) agonist, and an immunomodulatory monoclonal antibody and/or an immunosuppressive agent (e.g., an anti-CD3 antibody) increases β-cell function and/or increases insulin sensitivity in the population by at least about 4%, at least about 5%, at least about 6%, at least about 7%, at least about 8%, at least about 9%, at least about 10%, or more. Methods of measuring treatment-induced changes in β-cell function (such as β-cell proliferation) are well known in the art and include, e.g., hyperglycemic clamp, intravenous glucose tolerance test (IVGTT), graded glucose infusion, glucose-potentiated arginine stimulation, oral glucose tolerance test (OGTT) or mixed meal tolerance test (MMTT), and fasting measures (see, e.g., Hannon et al., “A Review of Methods for Measuring β-Cell function: Design Considerations from the Restoring Insulin Secretion (RISE) Consortium,” Diabetes Obes. Metab. 20(1):14-24 (2018), which is hereby incorporated by reference in its entirety). Methods of measuring treatment-induced changes in insulin sensitivity are well known in the art and include, e.g., hyperinsulinemic-euglycemic clamp, hyperglycemic clamp-derived insulin sensitivity, IVGTT—minimal model-derived insulin sensitivity (see, e.g., Hannon et al., “A Review of Methods for Measuring β-Cell function: Design Considerations from the Restoring Insulin Secretion (RISE) Consortium,” Diabetes Obes. Metab. 20(1):14-24 (2018), which is hereby incorporated by reference in its entirety).

In some embodiments, contacting a population of pancreatic beta cells with a dual-specificity tyrosine phosphorylation-regulated kinase 1A (DYRK1A) inhibitor, a glucagon-like peptide-1 receptor (GLP1R) agonist, and an immunomodulatory monoclonal antibody and/or an immunosuppressive agent (e.g., an anti-CD3 antibody) increases the number of proliferating pancreatic beta cells in the population by at least about 4%, at least about 5%, at least about 6%, at least about 7%, at least about 8%, at least about 9%, at least about 10%, or more.

In some embodiments, contacting a population of pancreatic beta cells with a dual-specificity tyrosine phosphorylation-regulated kinase 1A (DYRK1A) inhibitor, a glucagon-like peptide-1 receptor (GLP1R) agonist, and an immunomodulatory monoclonal antibody and/or an immunosuppressive agent (e.g., an anti-CD3 antibody) increases human beta cell mass by at least about 100%, at least about 110%, at least about 120%, at least about 130%, at least about 140%, at least about 150%, at least about 160%, at least about 170%, at least about 180%, at least about 190%, at least about 200%, at least about 210%, at least about 220%, at least about 230%, at least about 240%, at least about 250%, at least about 260%, at least about 270%, at least about 280%, at least about 290%, at least about 300%, at least about 350%, at least about 400%, at least about 450%, at least about 500%, at least about 550%, at least about 600%, at least about 650%, at least about 700%, or more. In some embodiments, contacting a population of pancreatic beta cells (e.g., human beta cells) with a dual-specificity tyrosine phosphorylation-regulated kinase 1A (DYRK1A) inhibitor, a glucagon-like peptide-1 receptor (GLP1R) agonist, and an immunomodulatory monoclonal antibody and/or an immunosuppressive agent (e.g., an anti-CD3 antibody) increases beta cell survival, as compared to when the population of pancreatic beta cells is not contacted.

In some embodiments, contacting a population of pancreatic beta cells with a dual-specificity tyrosine phosphorylation-regulated kinase 1A (DYRK1A) inhibitor, a glucagon-like peptide-1 receptor (GLP1R) agonist, and an immunomodulatory monoclonal antibody and/or an immunosuppressive agent (e.g., an anti-CD3 antibody) increases transdifferentiation of non-beta cells (e.g., alpha cells, delta cells, PP cells, and/or ductal cells) into beta-cells, as compared to when the population of pancreatic beta cells is not contacted.

In some embodiments, contacting a population of pancreatic beta cells with a dual-specificity tyrosine phosphorylation-regulated kinase 1A (DYRK1A) inhibitor, a glucagon-like peptide-1 receptor (GLP1R) agonist, and an immunomodulatory monoclonal antibody and/or an immunosuppressive agent (e.g., an anti-CD3 antibody) increases the number of proliferating pancreatic beta cells in a population by about 4-10% per day, or about 4-6% per day, 5-7% per day, 6-9% per day, or 7-10% per day.

In some embodiments, contacting a population of pancreatic beta cells with a dual-specificity tyrosine phosphorylation-regulated kinase 1A (DYRK1A) inhibitor, a glucagon-like peptide-1 receptor (GLP1R) agonist, and an immunomodulatory monoclonal antibody and/or an immunosuppressive agent (e.g., an anti-CD3 antibody) increases the number of proliferating pancreatic beta cells in a population by about 6-10% per day.

Methods of contacting a population of pancreatic beta cells with a dual-specificity tyrosine phosphorylation-regulated kinase 1A (DYRK1A) inhibitor, a glucagon-like peptide-1 receptor (GLP1R) agonist, and an immunomodulatory monoclonal antibody and/or an immunosuppressive agent (e.g., an anti-CD3 antibody) may be carried out under conditions effective to cause a synergistic increase in cell proliferation in a population of pancreatic beta cells, which means, inter alia, an increase in the number of proliferating pancreatic beta cells in the population as compared to when the cells are contacted with a DYRK1A inhibitor, a GLP1R agonist, or an immunomodulatory monoclonal antibody and/or an immunosuppressive agent (e.g., an anti-CD3 antibody).

In some embodiments, contacting a population of pancreatic beta cells with a dual-specificity tyrosine phosphorylation-regulated kinase 1A (DYRK1A) inhibitor, a glucagon-like peptide-1 receptor (GLP1R) agonist, and an immunomodulatory monoclonal antibody and/or an immunosuppressive agent (e.g., an anti-CD3 antibody) does not induce beta cell death or DNA damage in the population of cells. Moreover, contacting may induce beta cell differentiation and increase glucose-stimulated insulin secretion.

The method may be carried out to also enhance cell survival (in addition to reversing and/or restoring β-cell mass and function) in the population of pancreatic beta cells. For example, the method may be carried out to enhance cell survival of a treated population of pancreatic beta cells relative to an untreated population of pancreatic beta cells. Alternatively, the method may be carried out to decrease cell death or apoptosis of a contacted population of pancreatic beta cells relative to an uncontacted population of pancreatic beta cells.

### EXAMPLES

### Example 1—Materials and Methods

### Analysis of Human β-Cell Death In Vitro

Human islet cells were dispersed as previously described (Vasavada et al., “Tissue-Specific Deletion of the Retinoblastoma Protein in the Pancreatic β-Cell Has Limited Effects on β-Cell Replication, Mass, and Function,” Diabetes 56:57-64 (2007), which is hereby incorporated by reference in its entirety). Briefly, after washing the human islets with PBS twice, 200 μl pre-warmed Accutase (Corning) was added, and the tube was incubated at 37° C. for 10 minutes. Then, complete RPMI medium (RPMI with 5 mM D-glucose, 10% FBS, 100 units/ml penicillin, and 100 μg/ml streptomycin) was added, the sample was centrifuged at 1000 rpm for three min, the pellet was washed with PBS and centrifuged again at 1000 rpm for three min. The pellet was resuspended in complete RPMI medium and 50,000 cells/well were plated on 12-mm glass coverslips, placed in 24-well plates, and incubated at 37° C. and 5% CO₂ for 24 hours. Next, the following was added: saline, cytokines (50 units/mL IL-1β, 1,000 units/mL TNF-α, and 1,000 units/mL IFN-γ) (R&D Systems) or 500 nM thapsigargin (ER stress inducer, Sigma-Aldrich) (Mellado-Gil et al., “Disruption of Hepatocyte Growth Factor/c-Met Signaling Enhances Pancreatic Beta-Cell Death and Accelerates the Onset of Diabetes,” Diabetes 60:525-36 (2011); Lu et al., “Dextran Sulfate Protects Pancreatic β-Cells, Reduces Autoimmunity, and Ameliorates Type 1 Diabetes,” Diabetes 69:1692-1707 (2020), which are hereby incorporated by reference in their entirety) together with 10 nM exendin-4 (MedChemExpress) (E), 10 μM harmine-HCl (Robert DeVita Lab, ISMMS) (H), or the combination of exendin-4 and harmine (E+H) to the wells. Cells were incubated at 37° C. and 5% CO₂ for 24 hours. Then, cells were fixed in 4% paraformaldehyde and TUNEL (cell death marker) and insulin staining were performed using the DeadEnd Fluorometric TUNEL System (Promega), anti-proinsulin/C-peptide antibody (DSHB), and DAPI for nuclei detection. A minimum of 1,500 β-cells were examined per coverslip.

Human Islet Single Cell RNA Sequencing (scRNA-seq)

Sc-RNAseq analysis was performed on human islets treated with cytokines and harmine (H), Exendin-4 (E), and harmine+exendin-4 (E+H) (as above) at 37° C. and 5% CO₂ for 6 h. After treatment, human islets were washed twice with PBS and centrifuged at 300 rpm for three min. After removing the PBS, 200 μl pre-warmed Accutase was added and the islets were incubated at 37° C. for 10 minutes. Then, complete RPMI medium was added, the sample centrifuged at 1000 rpm for three min, and the pellet was washed with PBS. Cells were then resuspended in binding buffer (Miltenyi Biotec) and dead cell removal beads, the tube was incubated for 15 minutes at room temperature and the cell suspension applied onto the dead cell removal column (Miltenyi Biotec), which was attached to the MACS separator. Subsequently, the effluent that was centrifuged was collected and resuspended with 200 μl of 2% BSA and RNase inhibitor 200 U/ml in PBS, cells were mixed with AOPI (Nexcelon Bioscience) at 1:1 ratio and the cell concentration in the Countess 3 Automated Cell Counter (Thermo-Fisher) checked.

Cell samples were prepared according to the user guide of 10× Genomics Single Cell 3′ V3.1 Reagent Kit, processed with 10× Genomic Chromium Controller for partitioning and barcoding, followed by the cDNA library generation. The total cell concentration analyzed by the Countess 3 was then sequenced by the NovaSeq 6000 System (Illumina). The FASTQ files were downloaded from the sequencing facility and were aligned with Cell Ranger V.6.1.1 with Single Cell 3′ V3 chemistry on the 10× clouds pipeline. After the 10×, h5 format file was generated, the data were analyzed on the R language platform with Seurat package V.4.0. After the scRNA-seq data were created, ambient mRNA adjustment was performed with SoupX (20% contamination estimation). The cells were filtered out with less than 500 gene count, less than 250 gene varieties, less than 0.8 log₁₀ genes per UMI, and more than 20% mitochondrial gene ratio. Then, doublets were algorithmically removed with Doubltfinder package (20% estimation).

After assessment of the data quality control parameters as above, integrated scRNA-data were created with Seurat's SCTransform function without allocating method parameters. Then, the cell type was assigned identity according to the normalized gene expression level, referencing the canonical pancreatic cell type gene. To find changes in the apoptosis and pro-inflammatory pathways among the different treatments, single-cell level gene set enrichment analysis was performed in the β-cell population using the escape package, which accesses the entire Molecular Signature Database (v.7.0). The whole C2 library enrichment was executed with chemical and genetic perturbations and canonical pathways containing four well-known databases (Biocarta, KEGG, Reactome, and Wikipathways). After enrichment scores were calculated for each single cell, they were added to the Meta data for analysis and visualization by dotplot.

Treatment of Early-Onset Type 1 Diabetes (TID) Non-Obese Diabetic (NOD) Mice In Vivo with Anti-CD3 and Harmine+Exendin-4 (H+E)

Twelve- to sixteen-week-old NOD/LtJ (NOD) female mice (The Jackson Laboratory) were housed in specific pathogen-free conditions. Nonfasting blood glucose was measured once a week by a portable glucometer (AlphaTRAK 2; Abbott Laboratories); mice were considered diabetic when blood glucose was >250 mg/dL in three consecutive measurements in three consecutive days (Lu et al., “Dextran Sulfate Protects Pancreatic β-Cells, Reduces Autoimmunity, and Ameliorates Type 1 Diabetes,” Diabetes 69:1692-1707 (2020)). Then, early-onset diabetic mice were iv treated once daily for 3 days with 5 μg IgG or anti-CD3 antibody (non-Fc-binding monoclonal anti-CD3F F(ab′)2 obtained from Bio X Cell, https://bxcell.com/product/m-CD3e-fab2-fragments/). After the third injection, a minipump was implanted in the mouse interscapular region for continuous delivery of harmine, exendin-4, or harmine plus exendin-4 (Rosselot et al., “Human Beta Cell Mass Expansion In Vivo with a Harmine and Exendin-4 Combination: Quantification and Visulaization by iDISCO+3D Imaging,” biorxiv (2021), which is hereby incorporated by reference in its entirety). Briefly, harmine and exendin-4 were dissolved in water and loaded into Alzet (Cupertino, CA) model 1004 mini-osmotic pumps at a concentration of 27 mg/ml and 1 mg/ml, respectively, to permit subcutaneous delivery of harmine and exendin-4 for one month at a continuous rate of 3 mg/kg/day and 0.1 mg/kg/day, respectively. For two-month treatments, pumps were replaced at 28 days with new pumps and fresh harmine and exendin-4. Control pumps contained water. After pump implantation, nonfasting blood glucose was measured weekly as above and the percentage of diabetic mice was calculated. Animal studies were performed with the approval of and in accordance with guidelines established by the Icahn School of Medicine at Mount Sinai Institutional Animal Care and Use Committee.

Immune Cell Phenotyping of Splenocytes from Non-Obese Diabetic (NOD) Mice Treated with Anti-CD3 and Harmine and Exendin-4 (H+E)

At the end of the eight-week treatment, spleens were collected, grinded, and made into a cell suspension after lysis of the red blood cells and filtration (Lu et al., “Dextran Sulfate Protects Pancreatic β-Cells, Reduces Autoimmunity, and Ameliorates Type 1 Diabetes,” Diabetes 69:1692-1707 (2020), which is hereby incorporated by reference in its entirety). Cells (10⁶ cells/ml) were treated for 16 hours with 2 g/mL soluble anti-CD3 and 2 g/mL soluble anti-CD28 (BioLegend). Surface and intracellular staining of T cells for flow cytometry was achieved with APC anti-mouse CD45 (BioLegend), anti-CD44-PE (BioLegend), anti-mouse CD62L-Brilliant violet 605 (BioLegend), anti-CD8-FITC (eBioscience), anti-CD4-Pacific Blue (BioLegend), anti-IFN-7-phycoerythrin (eBioscience), anti-CD25-PerCP-Cy5.5 (BioLegend), and anti-FoxP3-phycoerythrin (eBioscience). Live/dead cells were identified by the Zombie NIR Fixable Viability Kit (BioLegend). Cells were analyzed in the Attune Nxt flow cytometer (Thermo-Fisher).

Histomorphometric Analysis of Pancreases Obtained from Non-Obese Diabetic (NOD) Mice Treated with Anti-CD3 and H+E

At the end of the eight-week treatment, pancreases were harvested and fixed overnight at room temperature in neutral-buffered formalin. Pancreases were then paraffin embedded and sectioned, and the β-cell mass was measured in three nonconsecutive insulin- and hematoxylin-stained sections per mouse using ImageJ (National Institutes of Health). Sections were also stained for Ki67 (Thermo-Fisher) or TUNEL (cell death, see above) and insulin (guinea-pig anti-insulin antibody, Abcam) to detect β-cell proliferation and death. Sections were also stained with hematoxylin and eosin for pathologic evaluation of islet insulitis that was calculated as percent of islets per mouse in each stage of insulitis (Lu et al., “Dextran Sulfate Protects Pancreatic β-Cells, Reduces Autoimmunity, and Ameliorates Type 1 Diabetes,” Diabetes 69:1692-1707 (2020), which is hereby incorporated by reference in its entirety).

### Statistical Analysis

Data presented as bar graphs, scatterplots and dotplots show means±SEM. Statistical significance analysis was performed using One-Way ANOVA (Tukey's post hoc test) or Student's t-test where appropriate for comparison among groups. P<0.05 was considered statistically significant.

### Example 2—Combination of Harmine with Exendin-4 Protects Human P-Cells from Inducers of Cell Death In Vitro

Proinflammatory cytokines and ER stress are established inducers of β-cell demise in Type 1 Diabetes (T1D) (Lu et al., “Dextran Sulfate Protects Pancreatic β-Cells, Reduces Autoimmunity, and Ameliorates Type 1 Diabetes,” Diabetes 69:1692-1707 (2020), which is hereby incorporated by reference in its entirety). Thus, it was first asked whether harmine (H), exendin-4 (E), or the combination (H+E) might have a protective effect on human j-cells when treated in vitro with pro-inflammatory cytokines or the ER stress inducer, thapsigargin. As shown in FIGS. 1A-1C, 10 μM harmine (H) and 10 nM exendin-4 (E) together significantly reduced human β-cell death induced by cytokines (FIGS. 1A-1B) or 500 nM thapsigargin (FIG. 1C) compared with 10 μM harmine or 10 nM exendin-4, which provided only non-significant, partial protection against cell death.

Single cell RNA sequencing (scRNA-seq) analysis of human islets treated with cytokines and harmine (H) and/or exendin-4 (E) as indicated above was next performed. It was found that pro-inflammatory (FIG. 1D), intrinsic and extrinsic apoptosis (FIG. 1E), human leukocyte antigens (HLA) class I molecules (FIG. 1F), chemokines CXCL9-11 (FIG. 1G), and interferon regulatory factor 1-9 (FIG. 1H) signaling pathways were upregulated in β-cells treated with cytokines and that treatment with the combination of harmine+exendin-4 (H+E) clearly downregulated expression of these pathways to levels approaching normal (FIGS. 1D-1H). Thus, treatment with harmine+exendin-4 (H+E) appears to provide protection to β-cells in a T1D environment.

### Example 3—Continuous Administration of Harmine Plus Exendin-4 Following Temporary Treatment with Anti-CD3 Completely Reverses Early-Onset Type 1 Diabetes (TID) in Non-Obese Diabetic (NOD) Mice

Based on the prosurvival and regenerative actions of harmine+exendin-4 in human β-cells (FIGS. 1A-1H; Ackeifi et al., “GLP-1 Receptor Agonists Synergize with DYRK1A Inhibitors to Potentiate Functional Human B Cell Regeneration,” Sci. Trans. Med. 12:eaaw9996 (2020) and Rosselot et al., “Human Beta Cell Mass Expansion In Vivo with a Harmine and Exendin-4 Combination: Quantification and Visulaization by iDISCO+3D Imaging,” biorxiv (2021), which are hereby Incorporated by reference in their entirety), and the promising but partial efficacy of anti-CD3 antibodies for treating early onset type 1 diabetes (T1D) (Herold et al., “Teplizumab (anti-CD3 mAb) Treatment Preserves C-Peptide Responses in Patients with New-Onset Type 1 Diabetes in a Randomized Controlled Trial: Metabolic and Immunologic Features at Baseline Identify a Subgroup of Responders,” Diabetes 62:3766-74 (2013); Herold et al., “Anti-CD3 Monoclonal Antibody in New-Onset Type 1 Diabetes Mellitus,” N. Engl. J. Med. 346:1692-8 (2002); Keymeulen et al., “Insulin Needs After CD3-Antibody Therapy in New-Onset Type 1 Diabetes,” N. Engl. J. Med. 352:2598-608 (2005); Sherry et al., “Teplizumab for Treatment of Type 1 Diabetes (Protégé Study): 1-Year Results from a Randomised, Placebo-Controlled Trial,” Lancet 378:487-97 (2011); Hagopian et al., “Teplizumab Preserves C-Peptide in Recent-Onset Type 1 Diabetes: Two-Year Results from the Randomized, Placebo-Controlled Protégé Trial,” Diabetes 62:3901-8 (2013); and Herold et al., “An Anti-CD3 Antibody, Teplizumab, In Relatives at Risk for Type 1 Diabetes,” N. Engl. J. Med. 381:603-613 (2019), which are hereby incorporated by reference in their entirety), it was tested whether the combination of harmine+exendin-4 (H+E) with anti-CD3 might be capable of reversing diabetes in early onset diabetic NOD mice.

Mice spontaneously developed diabetes (blood glucose above 250 mg/dl for three consecutive measurements) at 12-16 weeks of age, at which point they were iv treated once daily for 3 days with 5 μg of IgG or anti-CD3 antibody (non-Fc-binding monoclonal anti-CD3ε F(ab′)2 obtained from Bio X Cell, https://bxcell.com/product/m-CD3e-fab2-fragments/). Of note, this non-FcR-binding monoclonal anti-CD3 induces apoptosis of antigen-activated T-cells in vivo by allowing durable expression of the TCR and sustained signaling. Importantly, however, Foxp3+ Tregs have been shown to be resistant to CD3 antibody-mediated depletion. After the third injection, an Alzet minipump was implanted for continuous delivery of harmine, exendin-4, harmine and exendin-4 or water for four weeks.

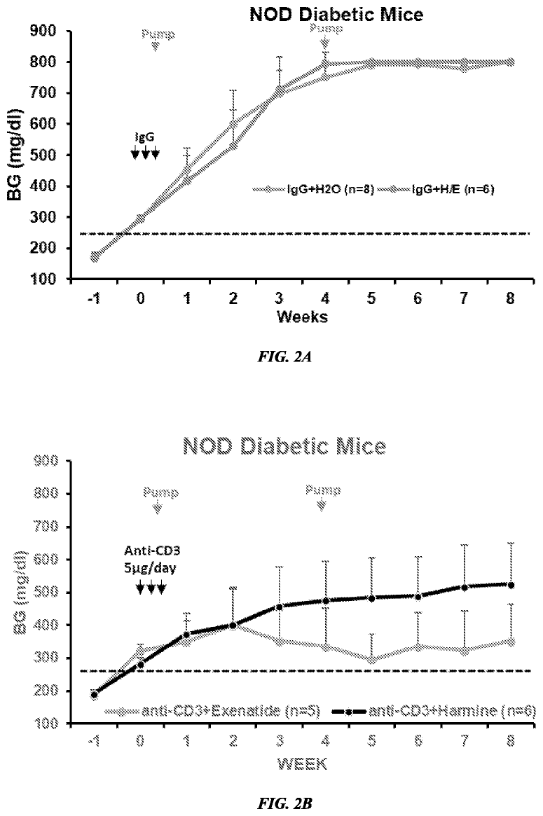

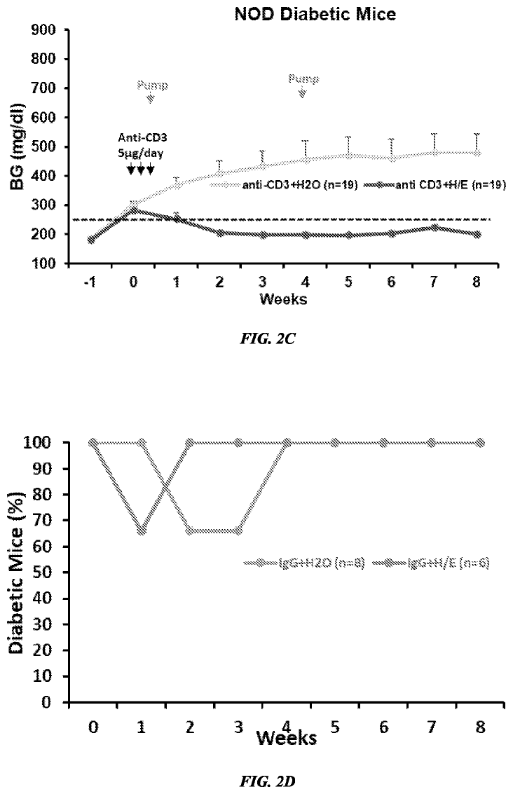

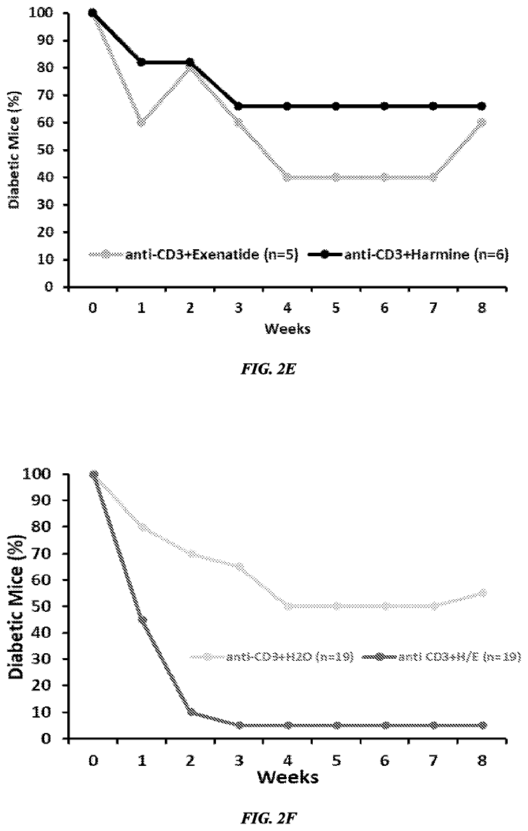

After four weeks, the minipump was replaced with a fresh one (Rosselot et al., “Human Beta Cell Mass Expansion In Vivo with a Harmine and Exendin-4 Combination: Quantification and Visulaization by iDISCO+3D Imaging,” biorxiv (2021), which is hereby incorporated by reference in its entirety) for a second four-week period. As shown in FIG. 2C, by two months after the intervention, harmine+exendin-4 treatment had reduced blood glucose levels to less than 250 mg/dl, while diabetic mice treated with vehicle remained diabetic during the eight-week follow-up period. Notably, 95% of mice treated with anti-CD3 and H+E remained euglycemic from week two to week eight (FIG. 2F). In contrast, only 40% of mice treated with anti-CD3 and vehicle remained euglycemic at week eight after treatment initiation (FIG. 2F). Of note, treatment of mice with 5 μg anti-CD3 per mouse per day for three days followed by either 3 mg/kg/day harmine or 0.1 mg/kg/day exendin-4 did not reduce blood glucose levels to less than 250 mg/dl (FIG. 2B) during the eight-week follow-up period; 70% of mice treated with anti-CD3 for three days followed by harmine remained diabetic from week 3 to week 8 of the eight-week follow-up period (FIG. 2E); and 40% of mice treated with anti-CD3 for three days followed by exendin-4 remained diabetic from week 4 to week 7 of the eight-week follow-up period, with 60% of mice remaining diabetic on week 8 of the eight-week follow-up period (FIG. 2E). No significant differences in blood glucose level were observed when mice were treated with 5 μg IgG per mouse per day for three days followed by either (i) 3 mg/kg/day harmine and 0.1 mg/kg/day exendin-4 or (ii) vehicle (H₂O) for eight weeks (FIG. 2A, FIG. 2D), with 70-100% of mice remaining diabetic in both groups during the eight-week follow-up period.

### Example 4—Immunophenotyping of Splenocytes in Early-Onset Type 1 Diabetes (T1D) Non-Obese Diabetic (NOD) Mice Treated with Anti-CD3 and H+E

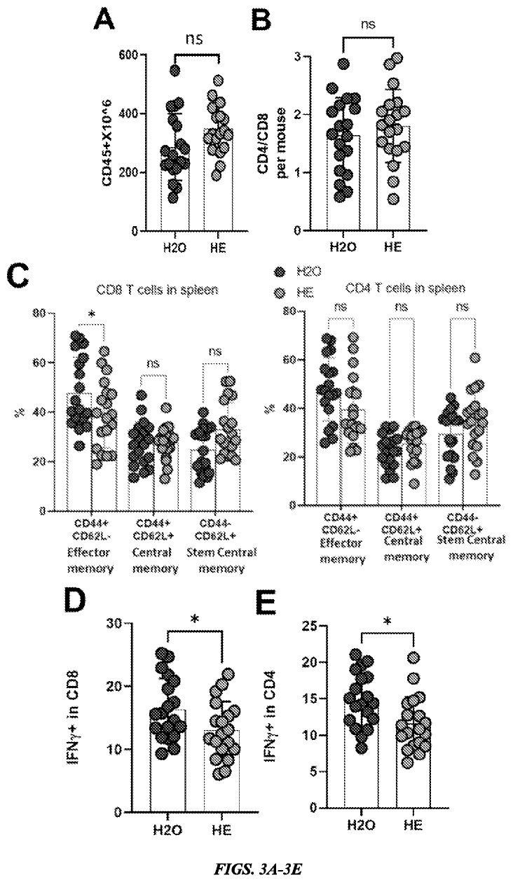

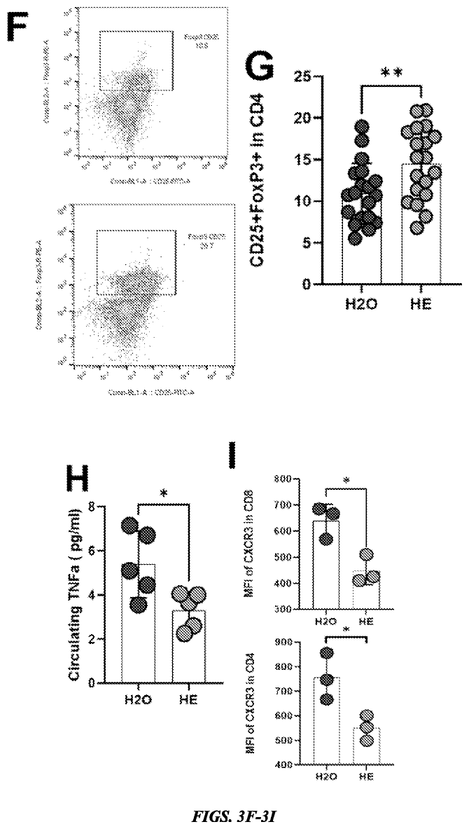

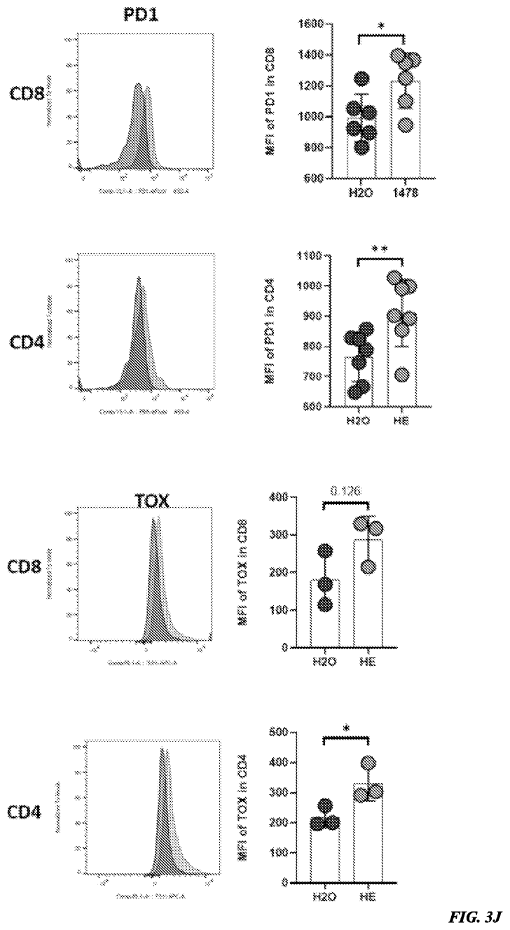

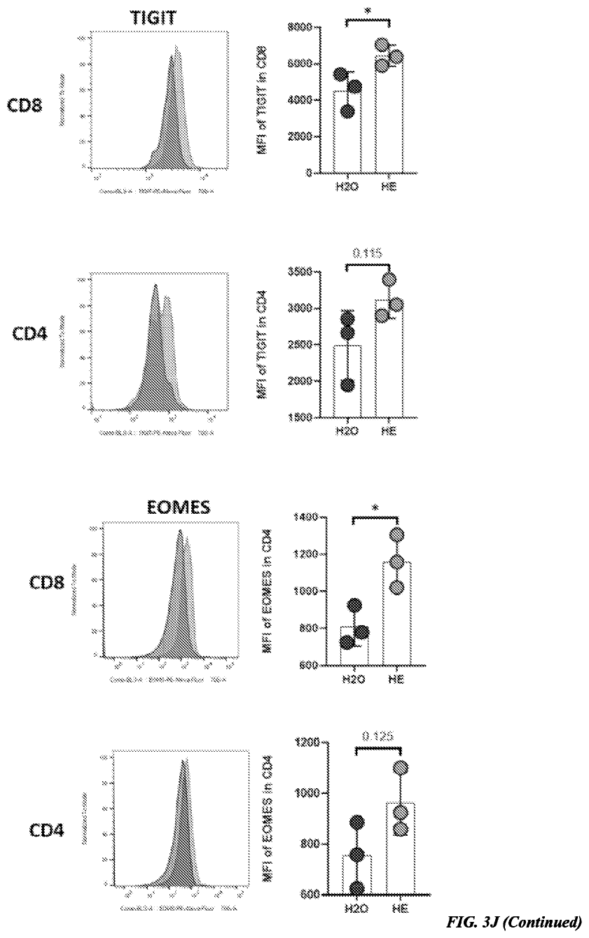

In ongoing studies, analysis of immune cell populations in blood, spleen, and pancreatic lymph nodes of early-onset T1D NOD mice treated with anti-CD3 and harmine+exendin-4 (H+E) has begun. Analysis of splenocytes at the end of the study (week 8) indicated that the total number of immune cells (CD45+, naive, memory and effector CD4+ and CD8+T lymphocytes) do not significantly change in the two treatment groups (FIGS. 3A-3C). Analysis of specific immune cell populations reveals that harmine+exendin-4 (H+E) treatment significantly decreases the number of activated CD4⁺ and CD8⁺ T-cells (Th1) (FIGS. 3D-3E) and significantly increases the number of FoxP3⁺ CD25⁺ regulatory T-cells (Tregs) by more than 50% (FIGS. 3F-3G). This suggests induction of immune tolerance (decrease in T cell activation and enhanced regulatory T cells) following treatment with anti-CD3 and the harmine+exendin-4 (H+E) combination. In studies where NOD mice were treated with anti-CD3 per day for three days and 3 mg/kg/day harmine (H) and 0.1 mg/kg/day exendin-4 (E) or vehicle (H₂O) for eight weeks, circulating TNF alpha levels (FIG. 3H), as well as CXCR3⁺ CD8⁺ (FIG. 3I, top) and CXCR3⁺ CD4⁺ (FIG. 3I, bottom) cells were lower in harmine+exendin-4 (H+E) treated mice than in mice treated with vehicle, whereas T cell exhaustion markers PD1, TIGIT, TOX, and EOMES were higher in CD4⁺ and CD8+ cells in NOD mice treated for three days with anti-CD3 and then for 2-weeks with harmine+exendin-4 (H+E), as compared to NOD mice treated for three days with anti-CD3 and then for 2-weeks with vehicle (water) (FIG. 3J).

### Example 5—Analysis of Pancreases from NOD Mice Treated with Anti-CD3 and H+E

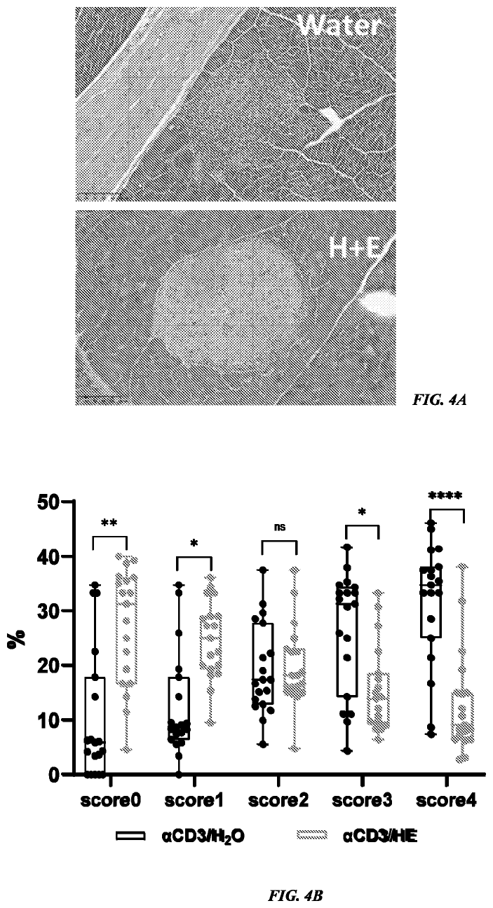

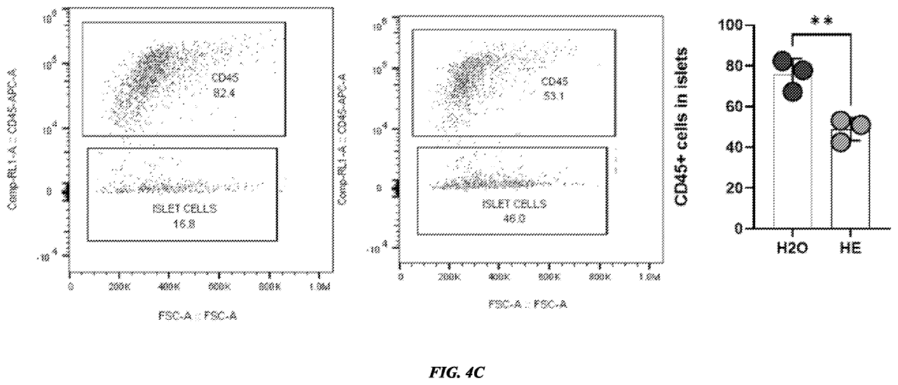

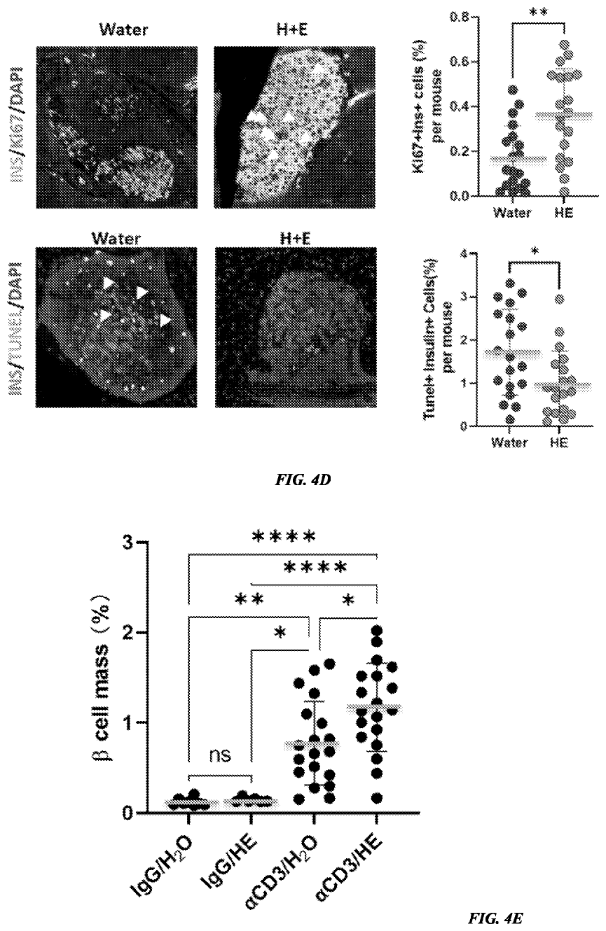

Hematoxylin & eosin staining of pancreatic sections obtained eight weeks after treatment with anti-CD3 and harmine+exendin-4 (H+E) or vehicle (FIGS. 4A-4B) was performed. It was immediately clear that islets from vehicle-treated mice contained the expected islet insulitis, and this was reduced in mice treated with anti-CD3 and harmine+exendin-4 (H+E). Insulitis scores for the islets in these pancreases reveal that mice treated with anti-CD3 and harmine+exendin-4 (H+E) have more islets with scores 0 to 2 (no insulitis to mild insulitis) while lower number of islets with strong and severe insulitis (score 3-4) compared with vehicle-treated mice (FIG. 4B). Thus, anti-CD3⁺ harmine+exendin-4 (H+E) treatment decreases islet inflammation in NOD diabetic mice. Flow cytometry analysis of islets from treated mice showed less CD45⁺ cells (immune cells) in the islets of mice treated with anti-CD3+ harmine+exendin-4 (H+E), as compared to mice treated with vehicle (FIG. 4C).

β-cell proliferation, β-cell death, and β-cell mass was next analyzed in these pancreases. As shown in FIGS. 4D-4E, Ki67+/insulin+cells were significantly increased while TUNEL+/insulin+were significantly decreased in mice treated with anti-CD3 and harmine+exendin-4 (H+E) compared with vehicle-treated mice, indicating an increase in β-cell proliferation and a decrease in β-cell death. In addition, analysis of total β-cell mass in these pancreases indicates that anti-CD3 and harmine+exendin-4 (H+E) treatment doubles the numbers of β-cells compared with vehicle-treated animals.

### CONCLUSION

Collectively, the studies described in these examples demonstrate for the first time that temporary immunomodulatory treatment with anti-CD3 followed by harmine+exendin-4 (H+E) combination treatment increases immune tolerance, enhances β-cell proliferation, protects β-cells and increases β-cell mass, effects that collectively lead to reversal of early onset T1D in NOD mice.

Although preferred embodiments have been depicted and described in detail herein, it will be apparent to those skilled in the relevant art that various modifications, additions, substitutions, and the like can be made without departing from the spirit of the invention and these are therefore considered to be within the scope of the invention as defined in the claims which follow.

What is claimed is:

1. A method of treating a subject for a condition associated with insufficient insulin secretion, said method comprising:
administering to a subject in need of treatment for a condition associated with an insufficient level of insulin secretion a dual-specificity tyrosine phosphorylation-regulated kinase 1A (DYRK1A) inhibitor, a glucagon-like peptide-1 receptor (GLP1R) agonist, and an immunomodulatory monoclonal antibody, optionally wherein the immunomodulatory monoclonal antibody is an anti-CD3 antibody;
wherein said administering is carried out under conditions effective to reverse loss of β-cell mass and function in the subject to treat the subject for the condition associate with insufficient insulin secretion.

2. The method according to claim 1, wherein the subject is treated for one or more of Type I diabetes (“T1D”), Type II diabetes (“T2D”), gestational diabetes, congenital diabetes, maturity onset diabetes (“MODY”), cystic fibrosis-related diabetes, hemochromatosis-related diabetes, drug-induced diabetes, or monogenic diabetes.

3. The method according to claim 2, wherein the subject is treated for Type I diabetes.

4. The method according to any one of claims 1-3, wherein the subject has long term Type 1 diabetes.

5. The method according to any one of claims 1-3, wherein the subject has recent onset Type 1 diabetes.

6. The method according to any one of claims 1-5, wherein said administering increases immune tolerance in the subject, enhances β-cell proliferation in the subject, protects β-cells in the subject, increases β-cell mass in the subject, and combinations thereof.

7. The method according to any one of claims 1-6, wherein the DYRK1A inhibitor is harmine.

8. The method according to any one of claims 1-7, wherein the GLP1R agonist is exendin-4.

9. The method according to any one of claims 1-8, wherein the anti-CD3 antibody is teplizumab.

10. The method according to any one of claims 1-9, wherein said administering is carried out with harmine, exendin-4, and teplizumab.

11. The method according to any one of claims 1-10, wherein said administering is carried out serially with each of the dual-specificity tyrosine phosphorylation-regulated kinase 1A (DYRK1A) inhibitor, the glucagon-like peptide-1 receptor (GLP1R) agonist, and the anti-CD3 antibody.

12. The method according to any one of claims 1-11, wherein said administering is carried out by first administering the anti-CD3 antibody.

13. The method according to claim 12, wherein said administering the anti-CD3 antibody is followed by treatment with the dual-specificity tyrosine phosphorylation-regulated kinase 1A (DYRK1A) inhibitor and the glucagon-like peptide-1 receptor (GLP1R) agonist.

14. The method according to any one of claims 1-13, wherein the anti-CD3 antibody is administered at a low dose.

15. The method according to any one of claims 1-14, wherein said administering is carried out nasally, orally, transdermally, parenterally, subcutaneously, intravenously, intramuscularly, or intraperitoneally.

16. The method according to any one of claims 1-15, wherein the subject is a mammalian subject.

17. The method according to any one of claims 1-16, wherein the subject is a human subject.

18. A composition comprising:
a dual-specificity tyrosine phosphorylation-regulated kinase 1A (DYRK1A) inhibitor;
a glucagon-like peptide-1 receptor (GLP1R) agonist; and
an immunomodulatory monoclonal antibody, optionally, wherein the immunomodulatory monoclonal antibody is an anti-CD3 antibody.

19. The composition according to claim 18 further comprising:
a carrier.

20. The composition according to claim 18 or claim 19, wherein the carrier is a pharmaceutically-acceptable carrier.

21. The composition according to any one of claims 18-20, wherein the DYRK1A inhibitor is harmine.

22. The composition according to any one of claims 18-21, wherein the GLP1R agonist is exendin-4.

23. The method according to any one of claims 18-22, wherein the anti-CD3 antibody is teplizumab.

24. A method of increasing β-cell mass and function in a population of pancreatic beta cells, said method comprising:
contacting a population of pancreatic beta cells with a dual-specificity tyrosine phosphorylation-regulated kinase 1A (DYRK1A) inhibitor, a glucagon-like peptide-1 receptor (GLP1R) agonist, and a low dose of an immunomodulatory monoclonal antibody, optionally wherein the immunomodulatory monoclonal antibody is an anti-CD3 antibody, wherein said contacting is carried out under conditions effective to increase β-cell mass and function in the population of pancreatic beta cells.

25. The method according to claim 24, wherein said method is carried out ex vivo.

26. The method according to claim 24, wherein said method is carried out in vivo.

27. The method according to any one of claims 24-26, wherein the DYRK1A inhibitor is harmine.

28. The method according to any one of claims 24-27, wherein the GLP1R agonist is exendin-4.

29. The method according to any one of claims 24-28, wherein the anti-CD3 antibody is teplizumab.

30. The method according to any one of claims 24-29, wherein said pancreatic beta cells are primary human pancreatic beta cells.
